(19) United States
(12) Patent Application Publication
(10) Pub. No.: US 2024/0318256 A1
(43) Pub. Date: Sep. 26, 2024

(54) METHOD TO DIAGNOSE MSI CANCER

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITÉ, Paris (FR); UNIVERSITÉ DE LILLE, Lille (FR); CENTRE HOSPITALIER RÉGIONAL UNIVERSITAIRE DE LILLE, Lille (FR)

(72) Inventors: Alex DUVAL, Paris (FR); Toky RATOVOMANANA, Paris (FR); Florence RENAUD, Paris (FR); Ada COLLURA, Paris (FR); Vincent JONCHERE, Paris (FR); Thierry ANDRE, Paris Cedex 12 (FR); Olivier BUHARD, Paris (FR); Florence COULET, Paris (FR)

(73) Assignees: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITÉ, Paris (FR); UNIVERSITÉ DE LILLE, Lille (FR); CENTRE HOSPITALIER RÉGIONAL UNIVERSITAIRE DE LILLE, Lille (FR)

(21) Appl. No.: 18/262,440

(22) PCT Filed: Jan. 28, 2022
(86) PCT No.: PCT/EP2022/052080

§ 371 Date: Jul. 21, 2023

(30) Foreign Application Priority Data
Jan. 29, 2021 (EP) 21305121.2

(51) Int. Cl. C12Q 1/6886

(52) U.S. Cl. CPC C12Q 1/6886; C12Q 2600/156

(57) ABSTRACT

A method of diagnosing an MSI cancer in a patient including extracting and sequencing DNA from a tumoral sample and if available from a normal sample and operate an analyse of MNRs. The method is based on the demonstration that the FDA-approved NGS-based diagnostic test for identifying MSI in mCRC and nmCRC gave inaccurate results when compared with the gold standard reference methods. Consequently, whole exome sequencing (WES) data from all samples was further analyzed to improve detection of the MSI genomic signal in CRC and other primary tumor types. This allowed identification of weaknesses and limits of MSISensor and the design and validation of a newly optimized algorithm, namely MSICare. The high accuracy of MSICare for the detection of MSI in CRC and non-CRC tumors should allow it to become a future reference test for assessing MSI in pan-cancer.

### FIELD OF THE INVENTION

The present invention relates to a method of diagnosing an MSI cancer in a patient in need thereof comprising notably extracting and sequencing DNA from a tumoral sample and if available from a normal sample and operate an analyse of MNRs.

### BACKGROUND OF THE INVENTION

The human tumor phenotype referred to as microsatellite instability (MSI) is associated with inactivating alterations in mismatch repair (MMR) genes. MSI was first reported in inherited tumors associated with Lynch syndrome. This is one of the most frequent cancer predisposition syndromes in humans and requires specific care and genetic counseling. MSI was later observed in sporadic colorectal cancer (CRC) and more rarely in other primary tumors (1-4). Tumors with MSI generally show a dense infiltration with cytotoxic T-cell lymphocytes (5). Recently, it was reported that MSI tumors and notably MSI CRC resist this hostile immune microenvironment by overexpressing immune checkpoint (ICK)-related proteins to allow immune-escape (6, 7). Furthermore, MSI status was shown to predict clinical benefit from ICK inhibitors (ICI) in patients with metastatic CRC (mCRC) (8-11). These observations have led to international guidelines recommending universal MSI/dMMR screening of all newly diagnosed CRC (12). There is also increasing evidence to support the evaluation of MSI status in all human tumors, regardless of the primary tissue of origin.

Several specialized cancer centers, including ours, have aimed to standardize and validate the accepted reference methods for testing MSI and dMMR in CRC, i.e. polymerase chain reaction (PCR)-based methods for MSI (13-15) and immunohistochemistry (IHC) for dMMR (see also 16 for review). In mCRC, we recently highlighted that misinterpretation of the results for MSI and/or MMR testing using these gold standard methods could account for most cases showing primary resistance to ICI (17). In the meantime, an alternative FDA-approved method based on next generation sequencing (NGS) technology was reported for MSI screening in pan-cancer, including CRC (18). This was based on the use of an algorithm, namely MSISensor, that analyzes sequence reads at designated microsatellite regions in tumor and paired normal samples and reports the percentage of unstable loci as a cumulative score in the tumor (19). However, the diagnostic performance of MSISensor has yet to be evaluated in patient cohorts where the MSI/dMMR status was already been established using the reference IHC and MSI-PCR methods, notably in the prospective setting of mCRC patients treated with ICI.

### SUMMARY OF THE INVENTION

In the present work, the aim of the inventors was therefore to evaluate the performance of MSISensor for the detection of MSI in dMMR/MSI mCRC from multicenter, prospective patients involved in clinical trials with ICI (NCT02840604 and NCT033501260). To avoid misdiagnoses, they have previously reassessed dMMR and MSI status in all CRC samples in an expert center using IHC and MSI-PCR. To further evaluate the results obtained with MSISensor in human cancers, they also analyzed a retrospective, multicenter series of mCRC and non-metastatic CRC (nmCRC), as well as a publicly available series of CRC and other primary tumor types that frequently display MSI. The present findings demonstrate that the FDA-approved NGS-based diagnostic test for identifying MSI in mCRC and nmCRC gave inaccurate results when compared with the gold standard reference methods. Importantly, this misdiagnosis included 3 mCRC patients that showed a positive response to ICI but would not have been treated if MSISensor alone had been used for MSI screening without reference to the IHC and MSI PCR methods. Consequently, whole exome sequencing (WES) data from all samples was further analyzed to improve detection of the MSI genomic signal in CRC and other primary tumor types. This allowed them to identify the weaknesses and limits of MSISensor and then to design and validate a newly optimized algorithm, namely MSICare. The high accuracy of MSICare for the detection of MSI in CRC and non-CRC tumors should allow it to become a future reference test for assessing MSI in pan-cancer.

Thus, the present invention relates to a method of diagnosing an MSI cancer in a patient in need thereof comprising notably extracting and sequencing DNA from a tumoral sample and if available from a normal sample and operate an analyse of MNRs. Particularly, the invention is defined by its claims.

### DETAILED DESCRIPTION OF THE INVENTION

The inventors have therefore developed a method which can be used when a tumoral sample from a patient is available and optionally when a normal sample from said patient is also available.

Thus, the invention relates to a method of diagnosing an MSI cancer in a patient in need thereof comprising i) extracting DNA from a tumoral sample and if available from a normal sample obtained from said patient, ii) sequencing a number (N) of mononucleotide repeats (MNR) sequences having a length of at least 12 nucleic acids in the DNA of the normal sample of said patient and the corresponding MNR in the DNA of the tumoral sample of said patient or sequencing a number (N) of mononucleotide repeats (MNR) in the tumoral sample and having a length of at least 12 nucleic acids in the corresponding normal sample, iii) calculating the ΔRatio for each MNR depending if the normal sample is available or not, iv) calculating the tumor purity (TP) for the tumor sample, v) calculating the ΔRatio adjusted, vi) obtaining a MSICare score by doing the ratio of the number of MNR with a ΔRatio adjusted mutated on the total number of ΔRatio of the MNR and, vii) concluding that the patient in need thereof has a MSI cancer when the MSICare score obtained at the step vi) is superior than a calculated threshold value.

In a particular aspect, the invention relates to a method of diagnosing an MSI cancer in a patient in need thereof comprising i) extracting DNA from a tumoral sample and if available from a normal sample obtained from said patient, ii) sequencing a number (N) of mononucleotide repeats (MNR) sequences having a length of at least 12 nucleic acids in the DNA of the normal sample of said patient and the corresponding MNR in the DNA of the tumoral sample of said patient or sequencing a number (N) of mononucleotide repeats (MNR) in the tumoral sample and having a length of at least 12 nucleic acids in the corresponding normal sample, iii) calculating the ΔRatio for each MNR depending if the normal sample is available or not, iv) calculating the tumor purity (TP) for the tumor sample, v) calculating the ΔRatio adjusted thanks to the TP calculated in the iv), vi) obtaining a MSICare score by doing the ratio of the number of MNR with a ΔRatio adjusted mutated on the total number of ΔRatio of the MNR and, vii) concluding that the patient in need thereof has a MSI cancer when the MSICare score obtained at the step vi) is superior than a calculated threshold value.

When a tumoral sample and a normal sample are available from a patient, the method of the invention is described as below.

Thus, a first embodiment of the invention relates to a method of diagnosing an MSI cancer in a patient in need thereof comprising i) extracting DNA from a tumoral and normal samples obtained from said patient, ii) sequencing a number (N) of mononucleotide repeats (MNR) sequences having a length of at least 12 nucleic acids in the DNA of the normal samples of said patient and the corresponding MNR in the DNA of the tumoral samples of said patients, iii) calculating the ΔRatio for each MNR, iv) calculating the tumor purity (TP) for the tumor sample, v) calculating the ΔRatio adjusted, vi) obtaining a MSICare score by doing the ratio of the number of MNR with a ΔRatio adjusted mutated on the total number of ΔRatio of the MNR and, vii) concluding that the patient in need thereof has a MSI cancer when the MSICare score obtained at the step vi) is superior than a calculated threshold value.

In this particular embodiment, the invention relates to a method of diagnosing an MSI cancer in a patient in need thereof comprising i) extracting DNA from a tumoral and normal samples obtained from said patient, ii) sequencing a number (N) of mononucleotide repeats (MNR) sequences having a length of at least 12 nucleic acids in the DNA of the normal samples of said patient and the corresponding MNR in the DNA of the tumoral samples of said patients, iii) calculating the ΔRatio for each MNR, iv) calculating the tumor purity (TP) for the tumor sample, v) calculating the ΔRatio adjusted thanks to the TP calculated in the iv), vi) obtaining a MSICare score by doing the ratio of the number of MNR with a ΔRatio adjusted mutated on the total number of ΔRatio of the MNR and, vii) concluding that the patient in need thereof has a MSI cancer when the MSICare score obtained at the step vi) is superior than a calculated threshold value.

According to the first embodiment of the invention, “sequencing a number (N) of mononucleotide repeats (MNR) sequences having a length of at least 12 nucleic acids in the DNA of the normal samples of said patient and the corresponding MNR in the DNA of the tumoral samples of said patients” denotes that for this method, only the MNR having a length of at least 12 nucleic acids in the DNA of the normal samples of the patient will be consider but for the tumoral samples, the corresponding MNR will be sequenced whatever their size. For example, for a specific locus, a MNR of 12 nucleic acids in the normal samples will be sequenced and in the tumoral samples, the corresponding MNR which will be also sequenced could have a length of 11 nucleic acids or 10 acids nucleic acids for example depending of the mutations.

According to the first embodiment of the invention, the number (N) of mononucleotide repeats (MNR) sequences having a length of at least 12 nucleic acids in the DNA of the normal samples will be taking to account in the method according to the invention if these repeats are covered by at least 20 mapping reads in both normal and tumoral samples.

As used herein, the term “ΔRatio” denotes the estimation of the number of reads in the tumoral sample compare to the normal sample and is calculated as following: for each MNR, the normalized number of reads in normal tissue is subtracted from the normalized number of reads in tumor tissue [ΔRatio=% Tumor−% Normal] for each MNR. For example, for a specific locus, if the read is 98 for a MNR of 20 A and 2 for a MNR of 19 A in a normal sample and 90 for a MNR of 20 A and 10 for a MNR of 19 A for a tumoral sample, the ΔRatio will be: 10−2=8 for the MNR 19 A.

Then, for all the loci, the MSI index will be the sum of the ΔRatio of each MNR.

As used herein, the “MSI index” or “MSI signal” or “MSIg” corresponding to the sum of ΔRatio values for all candidate MNR.

As used herein, the term “tumour purity (TP) for the tumour sample” denotes the estimation of the percentage of contamination for the tumour by normal tissue and is calculated according to the first embodiment of the invention as following: doing the median of the MSI index obtained for the MNRs equal or superior to 14 in the normal sample (=equal or superior to a length of 14 acids nucleic, see Jonchere et al. 2018) and the corresponding MNR in the DNA of the tumoral samples of said patients and covered by at least 20 reads in normal tissue and 30 reads for the tumor samples.

In some embodiment, from an MNR of 14 or 13, the mutational frequency of said MNR is high and therefore the determination of the score is more precise. In particular, the TP is sufficiently effective from an MNR of 14.

According to the invention, the MNR equal or superior of 14 can be in this loci: chr7: 121099908-121099923, chr2:119647053-119647067, chr2:44318095-44318110, chr1:100267651-100267665, chr1:214653467-214653482.

As use herein, the term “ΔRatio adjusted” means the normalisation of the ΔRatio thanks to the TP and is calculated as following: ΔRatio-adjusted=ΔRatio×Estimated TP for the tumor sample for a specific MNR. When the ΔRatio-adjusted for the MNR is <50% that's means that the MNR is wild type and when the ΔRatio adjusted is >50% that's mean that the MNR is mutated.

As used herein, the term “MSIcare score” denotes the ratio of the number of MNR with a ΔRatio adjusted mutated on the total number of ΔRatio of the MNR (=ΔRatio adjusted mutated and (+) ΔRatio adjusted not mutated). The result is a number between 0 and 100 (percentage). Depending of the number of analyses, the MSIcare cut-off for a MSI colorectal cancer can be 17%, 18%, 19%, 20%, 21%, 22%, 23%, 24% and 25% and more particularly is of 20% or 21%. According to the invention and for a colorectal cancer, when the MSIcare score will be below 21, particularly below 20, the colorectal cancer will not be a MSI colorectal cancer and when the MSIcare score will be above 21, particularly above 20, the colorectal cancer will be a MSI colorectal cancer.

In some embodiment, a pure MSI signal can be captured only by considering somatic deletion of at least 2 bp or more in this long MNR. In particular, at least 2 bp of difference between an MNR from a tumor sample and an MNR from a normal sample.

In a second embodiment of the invention, in the diagnostic of MSI cancer, the healthcare professionals may be faced with the fact that they do not have a normal sample available from the patient to perform the method of the invention. They can just have the tumoral sample from the patient suspected to have a MSI cancer. In this case, an identification of a normal polymorphic zone of the repeats can be done thanks to free database for each repeat. Then, from the tumoral sample, only mutated repeats observed outside this normal polymorphic zone will be considered. Outside this polymorphic zone, the number of reads in the normal sample will considered equal to zero. In this context, the several steps of the method of the invention, can be done taking this into account.

Thus, in some embodiment, the MSICare can be achieved only with the tumoral sample.

Thus, in this second embodiment, the invention also relates to a method of diagnosing an MSI cancer in a patient in need thereof comprising i) extracting DNA from a tumoral sample obtained from said patient, ii) sequencing a number (N) of mononucleotide repeats (MNR) in the tumoral sample and having a length of at least 12 nucleic acids in the corresponding normal sample, iii) evaluating the normal polymorphic zones for the MNR, iv) evaluating the mutated MNR appearing in the tumor sample only outside the normal polymorphic zone of each MNR; v) calculating the ΔRatio for each MNR obtained from the tumoral sample; vi) calculating the tumor purity (TP) for the tumor sample, vii) calculating the ΔRatio adjusted, viii) obtaining a MSICare score by doing the ratio of the number of MNR with a ΔRatio adjusted mutated on the total number of ΔRatio of the MNR and, ix) concluding that the patient in need thereof has a MSI cancer when the MSICare score obtained at the step viii) is superior than a calculated threshold value (see results “MSIcare diagnosis in CRC normal-sample-free solid tumor”).

In this particular embodiment, the invention also relates to a method of diagnosing an MSI cancer in a patient in need thereof comprising i) extracting DNA from a tumoral sample obtained from said patient, ii) sequencing a number (N) of mononucleotide repeats (MNR) in the tumoral sample and having a length of at least 12 nucleic acids in the corresponding normal sample, iii) evaluating the normal polymorphic zones for the MNR, iv) evaluating the mutated MNR appearing in the tumor sample only outside the normal polymorphic zone of each MNR; v) calculating the ΔRatio for each MNR obtained from the tumoral sample taking into account that the normalized number of reads in normal tissue is equal to zero outside each polymorphic zone; vi) calculating the tumor purity (TP) for the tumor sample, vii) calculating the ΔRatio adjusted, viii) obtaining a MSICare score by doing the ratio of the number of MNR with a ΔRatio adjusted mutated on the total number of ΔRatio of the MNR and, ix) concluding that the patient in need thereof has a MSI cancer when the MSICare score obtained at the step viii) is superior than a calculated threshold value.

In this particular embodiment, the invention also relates to a method of diagnosing an MSI cancer in a patient in need thereof comprising i) extracting DNA from a tumoral sample obtained from said patient, ii) sequencing a number (N) of mononucleotide repeats (MNR) in the tumoral sample and having a length of at least 12 nucleic acids in the corresponding normal sample, iii) evaluating the normal polymorphic zones for the MNR, iv) evaluating the mutated MNR appearing in the tumor sample only outside the normal polymorphic zone of each MNR; v) calculating the ΔRatio for each MNR obtained from the tumoral sample taking into account that the normalized number of reads in normal tissue is equal to zero outside each polymorphic zone; vi) calculating the tumor purity (TP) for the tumor sample, vii) calculating the ΔRatio adjusted thanks to the TP calculated in the vi), viii) obtaining a MSICare score by doing the ratio of the number of MNR with a ΔRatio adjusted mutated o on the total number of ΔRatio of the MNR and, ix) concluding that the patient in need thereof has a MSI cancer when the MSICare score obtained at the step viii) is superior than a calculated threshold value.

As used herein, the information concerning the “corresponding normal sample” or “analogous normal sample” is obtained from data base and particularly free data base referencing repeats of the genome.

As used herein, the term “repeat” denotes the number of nucleic acids (or nucleic bases) repeated for a specific locus. So the term “repeat” denotes a length of nucleic acids. For example, if the repeat is 12 for the nucleic acids A (adenine), this means that the nucleic acids A is repeated 12 time consecutively in a specific locus. According to the invention, as used herein, the term “repeat” as the same meaning than “microsatellite”.

As used herein, the term “mutated repeat” (or “mutated MNR”) denotes that the repeat is mutated (deletion or addition of one or several nucleic acids) compared to the normal repeat (find in normal sample). A repeat is mutated in a context of MSI cancer for example.

As used herein, the term “non-mutated repeat” (or “non-mutated MNR”) denotes that the repeat has no mutation (deletion or addition of one or several nucleic acids) compared to the normal repeat (find in normal sample).

As used herein, the term “polymorph” or “polymorphic repeat” denotes the different size of repeat that we can find in a sample. Thus, a “polymorphic zone” denotes the different repeats that we can find in a sample and a “normal polymorphic zone” denotes the different repeats that we can find in a normal sample (not mutated). For example, in a normal context (or MSS context), for a given repeat, said repeat could have a size of 15 or 16 nucleic acids. In a MSI context, the same repeat could have a size of 13, 14, 15 or 16 nucleic acids. Thus, in this example, the normal polymorphic zone for the normal sample will be between 15 and 16 and thus all repeat of 13 or 14 nucleic acids will be considered as mutated repeats and thus will be considered according to the second aspect of the invention. Thus and according to the second embodiment, the ΔRatio is calculated thanks to the same method describe above. For example, for the mutations which will be identified outside the polymorphic zone, the % Normal can be equal to zero (0) and thus the ΔRatio can be equal to % Tumor as described above.

According to the second embodiment of the invention, the number (N) of mononucleotide repeats (MNR) sequences having a length of at least 12 nucleic acids in the DNA of the normal samples will be taking to account in the method according to the invention if these repeats are covered by at least 20 mapping reads only in the tumor samples. According to the second embodiment of the invention, the tumour purity is calculated as following: doing the median of the MSI index obtained for the MNRs equal or superior to 14 (=equal or superior to a length of 14 acids nucleic) in the normal sample (obtained thanks to a database for example) and the corresponding MNR in the DNA of the tumoral samples of said patients and covered by at least 30 reads for the tumor samples.

As used herein, the term “mononucleotide repeats” (MNR) denotes that the repeat have just a repetition of a unique nucleic acid. For example, a mononucleotide repeat for the nucleic acid A and having a length of 12 means a repetition of the nucleic acid A 12 times consecutively.

As used herein, the terms “loci” relates to a specific, fixed position on a chromosome where a particular gene or genetic marker is located. The term loci encompasses the terms “MNR” or “marker”.

As used herein, the term “the number of repeat” denotes the number of time when a “repeat” of a specific length (for example 14 consecutive nucleic acids A) for a specific locus is repeated. Thus, “the number of repeat” also denotes the number of loci containing a given repeat.

As used the term “read” denotes a DNA fragment produced by a sequencer instrument which are a partial or exact copie of a locus (or of MNR or marker) to be sequenced and are used to determine the content and sequential order of its nucleic acids.

In a particular embodiment, the reads counts per locus after sequencing is between 10 and 5000, between 10 and 4000, particularly between 100 and 4000, particularly between 1000 and 3000 and more particularly between 1500 and 2500. Particularly, the reads counts per locus after sequencing is 20, 30, 40, 50, 100, 150, 200, 250; 300, 350 or 400.

As used herein, the term “N” denotes the number maximal of repeats of one specific sequencing assay which also corresponds to the number of loci (or makers) tested. This number can begin to 1 up to a large number.

In a particular embodiment, the sequencing according to the invention is done to a number of loci between 10 and 1000 000, between 10 and 10 000, particularly, between 100 and 10 000 and more particularly between 100 and 1000.

In some embodiment, this number is between 1 and 1000, particularly between 1 and 441, more particularly between 21 or more. Particularly, this number is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441.

In some embodiment, for a better robustness of the method, the number of repeats sequenced is at least 21.

In some embodiment, the more markers there are, the more the test is optimal and robust.

According to the methods of the invention, the sequencing of the mononucleotide repeats (MNR) can be done on the Whole Exome (WE) or in particular loci.

In some embodiment, a number of N repeat is studied and depending on the organ affected by the tumor, the number of N mutated can vary.

As used herein the term “sample” refers to any biological sample obtained from the patient that is liable to contain DNA, particularly germinal DNA and particularly cancerous DNA (DNA from cancerous cell). Typically, samples include but are not limited to solid sample (e.g. biopsy) or to body fluid samples, such as blood, plasma or serum.

In a particular embodiment, the sample is a normal sample or a tumor sample.

As used herein, the term “normal sample” refers to DNA from healthy tissue. In particular, the normal simple is the peripheral blood mononuclear cell (PBMC) or mucous.

According to the invention, “normal sample” means also “healthy sample” or “wild-type sample” that is to say a sample without mutations, obtained from non-cancerous cell or cancerous-cell (MSS cancer) compared to a tumoral sample which will contain mutations (MSI cancer).

As used herein, the term “tumor sample” refers to any biologic sample that contains tumor DNA, in particular circulating tumor DNA primary blood cells (PBCs). In a particular embodiment, the sample is tumor circulating cells or is tumor solid mass. In a particular embodiment, germinal DNA obtained from PBMCs or PBCs will be used to diagnose MSI cancer. In a particular embodiment, the sample can be frozen or not and the sample can come from primary tumor or metastatic tumor.

In some embodiment, the tumor sample corresponds to a solid tumor.

As used herein, the term “solid tumor” has its general meaning in the art and relates to an abnormal mass of tissue that usually does not contain cysts or liquid areas (e.g. biopsy). Solid tumors may be benign (not cancer), or malignant (cancer). Different types of solid tumors are named for the type of cells that form them. Examples of solid tumors are sarcomas and carcinomas.

In some embodiment, the tumor sample corresponds to a liquid tumor

As used herein, the term “liquid tumor” has its general meaning in the art and relates to a tumor that occurs in the blood, bone marrow or lymph nodes. Different liquid tumors include types of Leukaemia, Lymphoma and Myeloma.

As used herein, the term “MSI cancer” denotes that an instability is detected in at least 2 microsatellite markers. On the contrary, if instability is detected in one or no microsatellite marker, then said cancer is a “MSS cancer” This definition is valuable only if the diagnostic is done by the pentaplex method (see for example Suraweera N et al., Evaluation of tumor microsatellite instability using five quasimonomorphic mononucleotide repeats and pentaplex PCR. Gastroenterology. 2002 or Buhard O et al., Multipopulation analysis of polymorphisms in five mononucleotide repeats used to determine the microsatellite instability status of human tumors. J Clin Oncol. 2006). A “MSS cancer” denotes to a cancer having stable microsatellite. A “MSI cancer” refers to a cancer having microsatellite instable.

Particularly, the method of the invention is useful to distinguish a MSI cancer than a MSS cancer.

In some embodiment, the MSIcare can be done for the MSI diagnostic regardless of the deficient protein.

In particular, the MSIcare can be used in a context where genes like MLH1, MSH2, MSH6 or PMS2 are deficient (e.g. mutated or non-functional).

As used herein, the term “patient” or ‘subject” denotes a mammal. Typically, a patient according to the invention refers to any subject (particularly human) afflicted with a MSI cancer.

As used herein the term “nucleic acid” or “nucleic base” has its general meaning in the art and refers to refers to a coding or non-coding nucleic sequence. Nucleic acids include DNA (deoxyribonucleic acid) and RNA (ribonucleic acid) nucleic acids. Example of nucleic acid thus include but are not limited to DNA, mRNA, tRNA, IRNA, tmRNA, miRNA, piRNA, snoRNA, and snRNA. Nucleic acids thus encompass coding and non-coding region of a genome (i.e. nuclear or mitochondrial).

In a particular embodiment, the lengths (x) (or number) of nucleic acids in a specific repeat is between 12 and 30 and particularly 12 and 18. According to the invention the lengths (x) (or number) of nucleic acids in a specific repeat can be 12, 13, 14, 15, 16, 17, 18, 19, 20, 21, 22, 23, 24, 25, 26, 27, 28, 29 or 30.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; Lynch syndrome (known as hereditary nonpolyposis colorectal cancer (HNPCC) syndrome) and CMMRD (constitutional mismatch repair deficiency) syndrome. In some embodiments, the patient suffers from a colorectal cancer, more particularly a metastatic colorectal cancer.

In a particular embodiment, the cancer is a metastatic cancer.

In a particular embodiment, the cancer is a colorectal cancer, a gastric cancer or an endometrial cancer.

In a particular embodiment, the cancer is a metastatic colorectal cancer, a metastatic gastric cancer or a metastatic endometrial cancer.

In one embodiment, a further step of communicating the result to the patient may be added to the methods of the invention.

In a particular embodiment, the methods as described above allows to distinguish a MSS cancer to a MSI cancer

According to the invention, the methods are ex-vivo methods or in-vitro methods.

The MSICare of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

Another object of the present invention is a computer-program product comprising code instructions for executing the method described above, when it is implemented by a computer.

Predetermined reference values used for comparison of the MSIcare score may comprise “cut-off” or “threshold” values that may be determined as described herein. Each reference (“cut-off”) value for MSIcare score level may be, for example, predetermined by carrying out a method comprising the steps of:
a) providing a collection of samples from patients suffering of a cancer;
b) determining the MSIcare score for each sample contained in the collection provided at step a);
c) ranking the tumor tissue samples according to said level
d) classifying said samples in pairs of subsets of increasing, respectively decreasing, number of members ranked according to their expression level,
e) providing, for each sample provided at step a), information relating to the actual clinical outcome for the corresponding cancer patient;
f) for each pair of subsets of samples, obtaining a Kaplan Meier percentage of survival curve;
g) for each pair of subsets of samples calculating the statistical significance (p value) between both subsets
h) selecting as reference value for the level, the value of level for which the p value is the smallest.

For example the MSIcare score has been assessed for 100 pancreatic cancer samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding pancreatic cancer patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate pancreatic cancer samples and therefore the corresponding patients.

Kaplan-Meier curves of percentage of survival as a function of time are commonly used to measure the fraction of patients living for a certain amount of time after treatment and are well known by the man skilled in the art.

The man skilled in the art also understands that the same technique of assessment of the expression level of a protein should of course be used for obtaining the reference value and thereafter for assessment of the expression level of a protein of a patient subjected to the method of the invention.

According to the invention, the MSICare cut-off was determined in an initiation cohort by choosing the rounded value where the maximum of the sum of sensitivity and specificity is obtained (Receiver Operating Characteristic (ROC) analysis). Depending of the number of analyses, the MSIcare cut-off for the MSI colorectal cancer can be between 17%18%, 19%, 20%, 21%, 22%, 23%, 24% and 25% and more particularly is of 20% or 21%. To do this, among the different usable methods the inventors applied the ‘cutpointr’ package (https://github.com/thiele/cutpointr) which estimate cutpoints that optimize a specified metric in binary classification tasks and validate performance using bootstrapping. As used herein, «specific metric» denotes the sum of sensitivity and specificity.

Another aspect of the invention relates to a method of diagnosing a mutation in a MNR in a patient in need thereof comprising i) extracting DNA from a tumor and normal samples obtained from said patient, ii) sequencing a number (N) of mononucleotide repeats (MNR) sequences having a length of at least 12 nucleic acids in the DNA of the normal samples of said patient and the corresponding MNR in the DNA of the tumoral samples of said patients, iii) calculating the ΔRatio for each MNR, iv) calculating the tumor purity (TP) for the tumor sample, v) calculating the ΔRatio adjusted and vi) concluding that the MNR is wild type when the ΔRatio-adjusted is <50% concluding that the MNR is mutated when the ΔRatio adjusted is ≥50%.

According to this particular aspect, of the invention also relates to a method of diagnosing a mutation in a MNR in a patient in need thereof comprising i) extracting DNA from a tumor and normal samples obtained from said patient, ii) sequencing a number (N) of mononucleotide repeats (MNR) sequences having a length of at least 12 nucleic acids in the DNA of the normal samples of said patient and the corresponding MNR in the DNA of the tumoral samples of said patients, iii) calculating the ΔRatio for each MNR, iv) calculating the tumor purity (TP) for the tumor sample, v) calculating the ΔRatio adjusted thanks to the TP calculated in the iv) and vi) concluding that the MNR is wild type when the ΔRatio-adjusted is <50% concluding that the MNR is mutated when the ΔRatio adjusted is ≥50%.

According to this method, a mutation is responsible of the apparition a cancerous cell.

In one embodiment, the mutation is a repeat or a microsatellite responsible of the apparition of an MSI cancer.

### Sequencing Methods:

According to the invention, the sequencing step may be accomplished by any method, including without limitation chemical sequencing, using the Maxam-Gilbert method (Methods in Enzymology 65, 499-560 (1980)); by enzymatic sequencing, using the Sanger method Proc. Natl. Acad. Sci. USA 74, 5463-67 (1977)).; mass spectrometry sequencing; sequencing using a chip-based technology; and real-time quantitative PCR.

In the chemical sequencing, base specific modifications result in a base specific cleavage of the radioactive or fluorescently labeled DNA fragment. With the four separate base specific cleavage reactions, four sets of nested fragments are produced which are separated according to length by polyacrylamide gel electrophoresis (PAGE). After autoradiography, the sequence can be read directly since each band (fragment) in the gel originates from a base specific cleavage event. Thus, the fragment lengths in the four “ladders” directly translate into a specific position in the DNA sequence.

In the enzymatic sequencing, the four base specific sets of DNA fragments are formed by starting with a primer/template system elongating the primer into the unknown DNA sequence area and thereby copying the template and synthesizing a complementary strand by DNA polymerases, such as Klenow fragment of E. coli DNA polymerase I, a DNA polymerase from Therm us aquaticus, Taq DNA polymerase, or a modified T7 DNA polymerase, Sequenase (Tabor et al., Proc. Natl. Acad. Scl. USA 84, 4767-4771 (1987)), in the presence of chain-terminating reagents.

Several new methods for DNA sequencing (High-throughput sequencing (HTS) methods) were developed in the mid to late 1990s and were implemented in commercial DNA sequencers by the year 2000. Together these were called the “next-generation” or “second-generation” sequencing methods. These HTS included but are not limited to: Single-molecule real-time sequencing, Ion semiconductor, Pyrosequencing, Sequencing by synthesis, Sequencing by ligation, Nanopore Sequencing, Chain termination and Sequencing by hybridization. Some of these methods allow a Whole Gene Sequencing (WGS), Whole Exome Sequencing (WES) or a Targeted Sequencing.

In a particular embodiment, the sequencing according to the method of the invention is an ultra-deep sequencing like Second-Generation Sequencing (NGS), performed using targeted massive parallel sequencing approcah, by the mean of which a specified panel of regions in the genome, herein mononucleoid microsatellites, are sequenced (see for example Goodwin, S and all, 2016. Coming of age: Ten years of next-generation sequencing technologies. Nature Reviews Genetics).

### Method of Treatment

In another aspect, the invention also relates to a method for treating a cancer in a patient identified has having a MSI cancer according to the methods of the invention comprising administering to said patient a therapeutically effective amount of radiotherapy, chemotherapy, immunotherapy or a combination thereof.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

When it is concluded that the patient has a MSI cancer then the physician can take the choice to administer the patient with a targeted therapy.

Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

When it is concluded that the patient has a MSI cancer then the physician can take the choice to administer the patient with an immunotherapeutic agent.

The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the patient with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-beta) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer.

Interleukins have also shown good activity in combination with IFN-alpha in the treatment of various cancers (Negrier et al., Ann Oncol. 2002 13(9): 1460-8; Touranietal, J. Clin. Oncol. 2003 21(21):398794).

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), Calmette-Guerin bacillus (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumours. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies.

Particularly, a patient diagnosed as having a CMMRD or a MSI leukemia/lymphoma according to the invention can be treated by immunotherapy like immune checkpoint blockade involving anti-CTLA4, anti-PD1, anti-PD-L1 alone or in combination, or anti-cancer vaccines or dendritic cells vaccines based on tumour specific antigens.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

The immunotherapeutic treatment may consist of an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, or tumor infiltrated lymphocytes, are isolated in vitro, activated by lymphokines such as IL-2 or transuded with genes for tumor necrosis, and readministered (Rosenberg et al., 1988; 1989). The activated lymphocytes are most preferably be the subject's own cells that were earlier isolated from a blood or tumor sample and activated (or “expanded”) in vitro. This form of immunotherapy has produced several cases of regression of melanoma and renal carcinoma.

When it is concluded that the patient has a MSI cancer then the physician can take the choice to administer the patient with a radiotherapeutic agent.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

### Kits or Devices of the Present Invention:

A further object of the present invention relates to a kit or device for performing the methods of the present invention, comprising means for extracting and sequencing DNA from a sample.

In some embodiments, the kit or device comprises at least one couple of primer per locus.

The invention will be further illustrated by the following figures and examples. However, these examples and figures should not be interpreted in any way as limiting the scope of the present invention.

### FIGURES

FIG. 1. Background and Study Design. FDA, Food and Drug Administration; MSI, microsatellite instable; CRC, colorectal cancer; mCRC, metastatic colorectal cancer; nmCRC, non-metastatic colorectal cancer; WES, whole exome sequencing; ICI, immune checkpoint inhibitors; IHC, Immunohistochemistry.

FIG. 2. Reassessment of MSI using MSISensor in prospective and retrospective cohorts of metastatic and non-metastatic CRC whose MSI/dMMR or MSS/pMMR status had been previously assessed with gold standard reference methods.
A) Boxplots show the percentage of mutated microsatellites (MSISensor score) obtained from WES of 25 MSI (red) and 77 MSS (blue) patients with metastatic CRC from a prospective cohort (Cohort 1, C1).
B) Boxplots show the percentage of mutated microsatellites (MSISensor score) obtained from WES of 88 MSI patients with non-metastatic CRC (left) and 25 MSI patients with metastatic CRC (right) from a retrospective cohort (Cohort 2, C2).
C) Boxplots show the percentage of mutated microsatellites (MSISensor score) obtained from WES of 118 TCGA patients, including 51 MSI, 14 MSI-L and 53 MSS patients (Cohort 3, C3).

A cutoff MSISensor score of 10 (FDA recommendation) was used to discriminate MSS from MSI tumors (green dotted line). Non-metastatic samples are represented by a circle and metastatic samples by a diamond. Horizontal barplots indicate the percentage of true negative (TN), true positive (TP), false negative (FN) and false positive (FP) for each cohort.

FIG. 3. Improving the computational detection of MSI in CRC by identifying the weaknesses and limits of MSISensor.

Density plot of the MSISensor score in C1+C2 (left) and C3 (right) cohorts. The cutoff MSISensor score of 10 (FDA recommendation) was used to separate MSS from MSI tumors (green dotted line). The adjacent histograms represent the distribution of tumor samples according to their MSISensor score.

FIG. 4. Testing the diagnostic performance of MSICare.
A) Density plot of the MSICare score in the C1 and C2 cohorts.
B) Density plot of the MSICare score in the C3 cohort.

FIG. 5. Comparative performance of MSISensor and MSICare for the identification of MSI in gastric and endometrial tumors from the TCGA.
A) Boxplots show the MSISensor score obtained from WES of 104 TCGA GC patients (including 55 MSI, 9 MSI-L and 42 MSS) and 278 TCGA EC patients (including 159 MSI, 17 MSI-L and 102 MSS) from Cohort 3. A cutoff MSISensor score of 10 (FDA recommendation) was used to discriminate MSS from MSI tumors (green dotted line).
B) Boxplots show the MSICare score obtained from WES of the same TCGA patients with CRC, GC or EC. A cutoff MSICare score of 20 was used to discriminate MSS from MSI tumors (green dotted line).

FIG. 6. Identification of weaknesses and limits of MSISensor for detecting MSI (lack of sensitivity).
A) Distribution of the total number of somatic mutations identified with MSISensor from WES data (C1 and C2) for each category of microsatellites (Mono-, Di-, Tri-, Tetra- or Penta-nucleotide repeats). Mononucleotide repeat (MNR) sequences are by far the most unstable category of microsatellites in dMMR colon tumors and are therefore better at distinguishing MSI from MSS CRC than other forms of repeats used by MSISensor (e.g. di-, tri-, tetra-, penta-).
B) Percentage of mutated MNR according to their size (range from 5 to 12, MSICare score) (C1 and C2). 5+, includes all mononucleotide repeats with a length of ≥5 bp; 6+, includes all mononucleotide repeats with a length of ≥6 bp, etc. The long MNR of ≥12 bp was best at discriminating the two phenotypes (Blue rectangle).

FIG. 7. Genomic instability Index according to MSISensor score. Distribution of genomic instability Index (based on mononucleotide repeat instability, see Materials and Methods for details) of tumors according to MSISensor score (x-axis) of cohorts C1 and C2

FIG. 8. Identification of the weaknesses and limits of MSISensor for detecting MSI (lack of specificity).
A) Density plot of reads according to the repeat length for 3 examples of Loss Of Heterozygosity (LOH) in 3 tumors identified as having mutated microsatellites by MSISensor.
B) Wild-type and mutated profiles of the T16 microsatellite are shown (normal DNA, wild-type, in green; MSI tumor DNA, mutated, in yellow). This illustrates that, due to stuttering (T15), a pure MSI signal can be captured only by considering somatic deletion of 2 bp or more in this long MNR.

FIG. 9. Comparative Performance of MSISensor and MSICare for the identification of MSI using targeted panel sequencing data
A) Boxplots show the percentage of mutated microsatellites (MSISensor score) obtained from the MSK-IMPACT for patients from the C1 and C2 cohorts (all patients, right panel; the same patients with either MLH1, MSH2, MSH6 or PMS2 deficient CRC, left panel). A cutoff MSISensor score of 10 (FDA recommendation) was used to discriminate MSS from MSI tumors (dotted line).
B) Boxplots show the percentage of mutated microsatellites (MSICare score) obtained from the MSK-IMPACTTM for patients from the C1 and C2 cohorts (all patients, right panel; the same patients with either MLH1, MSH2, MSH6 or PMS2 deficient CRC, left panel). A cutoff MSICare score of 20 was used to discriminate MSS from MSI tumors (dotted line).
C) Boxplots show the percentage of mutated microsatellites (MSISensor score and/or MSICare score) obtained following targeted sequencing of patients from the C4 cohort (all patients, right panel; the same patients with either MLH1, MSH2, MSH6 or PMS2 deficient CRC, left panel). The pentaplex profile of the only one misdiagnosed case is shown in a box. Horizontal barplots indicate the percentage of true negative (TN), true positive (TP), false negative (FN) and false positive (FP).

FIG. 10. Assessment of MSISensor score following targeted sequencing with MSIDIAG.

Boxplots show the percentage of mutated microsatellites (MSISensor score) obtained following targeted sequencing of patients from the C4 cohort (all patients, left panel; the same patients with either MLH1, MSH2, MSH6 or PMS2 deficient CRC, right panel).

Horizontal barplots indicate the percentage of true negative (TN), true positive (TP), false negative (FN) and false positive (FP).

FIG. 11. MSI level evaluation in brain tumor

Barplots of the percentage of mutated microsatellites (MSICare score) obtained from panel sequencing from WES of 8 MMRp (according to IHC) patients and 24 dMMR (according to IHC) patients with brain tumour. Four of the dMMR patient display Constitutional Mismatch Repair Deficiency CMMRD, 3 have a Lynch Syndrome and 17 have a dMMR tumour after Temozolomide treatment (post_TMZ). The y axis have a log 10 scale.

FIG. 12. Diagnosis of MSI status in CRC normal-sample-free solid tumor using WIND-MSICare

Boxplots show the percentage of mutated microsatellites (WIND-MSICare score) obtained from tumor only samples following targeted sequencing. Samples were either MMRd or MMRp according to IHC.

FIG. 13. Diagnosis of MSI status in tumor circulating DNA using WIND-MSICare

Barplots show the percentage of mutated microsatellites (WIND-MSICare score) obtained from panel sequencing from circulating DNA of 4 patients without any normal comparison. “T1” Sample was extracted the day of the first immunotherapy treatment. “T2” Sample was extracted 3 months after the first immunotherapy treatment. The patient MS-CIRC-041 is known to be pMMR according to IHC. The patients MS-CIRC-005,MS-CIRC-012 and MS-CIRC-045 are known to be dMMR according to IHC. A cutoff MSICare score of 20 was used to discriminate MSS from MSI tumors.

TABLE 1
Sensitivity, specificity, NPV, PPV of microsatellite instability
detection according to NGS methods on different cohort.

| | | | |
|---|---|---|---|
| Sensitivity % | Specificity % | PPV % | NPV % |
| (95% CI) | (95% CI) | (95% CI) | (95% CI) |

A. Estimate of the sensitivity, specificity, negative and positive predictive
values of microsatellite instability detection using MSIsensor.
Method: MSISensor

| | | | | |
|---|---|---|---|---|
| C1 | 84.0 | 100.0 | 100.0 | 95.1 |
| | (69.3-98.7) | (100.0-100.0) | (100.0-100.0) | (90.2-100.0) |
| C2 ALL | 85.8 | NA | NA | NA |
| | (79.3-92.3) | | | |
| C2 nmCRC | 90.9 | NA | NA | NA |
| | (84.9-96.9) | | | |
| C2 mCRC | 68.0 | NA | NA | NA |
| | (49.3-86.7) | | | |
| C1 + C2 | 85.5 | 100.0 | 100.0 | 79.4 |
| | (79.6-91.4) | (100.0-100.0) | (100.0-100.0) | (70.3-88.5) |
| C3 | 90.2 | 95.5 | 93.9 | 92.8 |
| | (82.0-98.4) | (90.5-100.5) | (87.3-100.5) | (86.5-99.1) |

B. Estimate of the sensitivity, specificity, negative, and positive predictive
values of microsatellite instability detection using MSIcare.
Method: MSICare

| | | | | |
|---|---|---|---|---|
| C1 | 100.0 | 100.0 | 100.0 | 100.0 |
| | (100.0-100.0) | (100.0-100.0) | (100.0-100.0) | (100.0-100.0) |
| C2 ALL | 100.0 | NA | NA | NA |
| | (100.0-100.0) | | | |
| C2 nmCRC | 100.0 | NA | NA | NA |
| | (100.0-100.0) | | | |
| C2 mCRC | 100.0 | NA | NA | NA |
| | (100.0-100.0) | | | |
| C1 + C2 | 100.0 | 100.0 | 100.0 | 100.0 |
| | (100.0-100.0) | (100.0-100.0) | (100.0-100.0) | (100.0-100.0) |
| C3 | 96.1 | 97.0 | 96.1 | 97.0 |
| | (90.7-101.5) | (92.9-101.1) | (90.7-101.5) | (92.9-101.1) |

CI, confidence interval
PPV, Positive predictive value
NPV, Negative predictive value
nmCRC, non-metastatic colorectal cancer
mCRC, metastatic colorectal cancer

### Example

### Material & Methods

### Study Populations

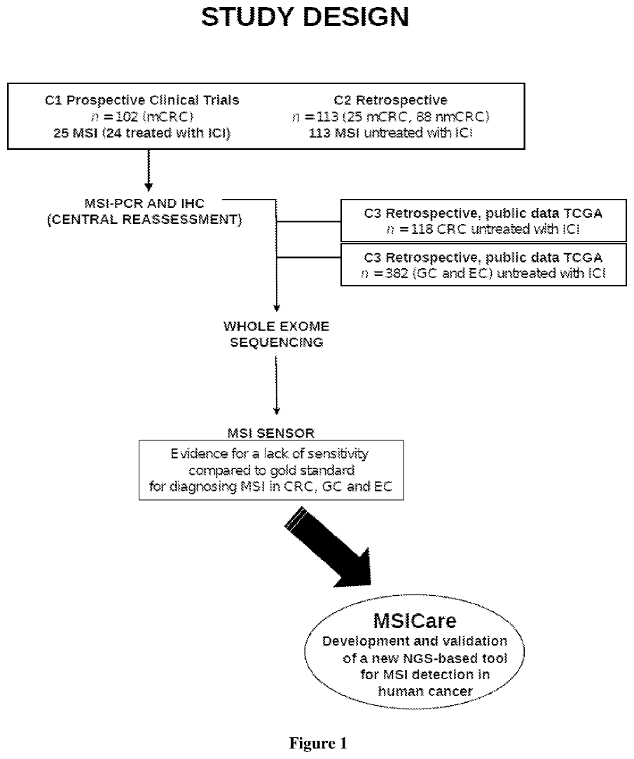

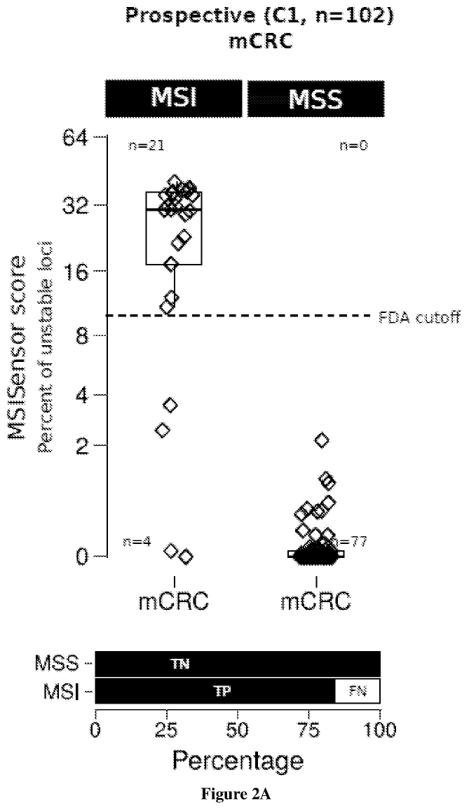

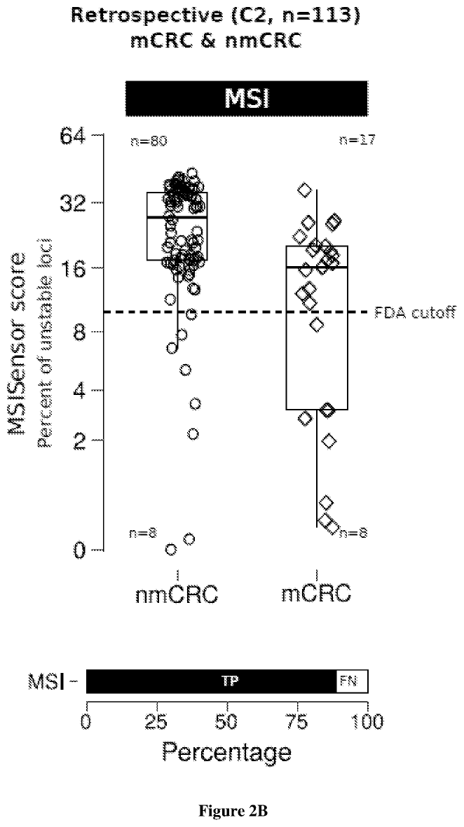

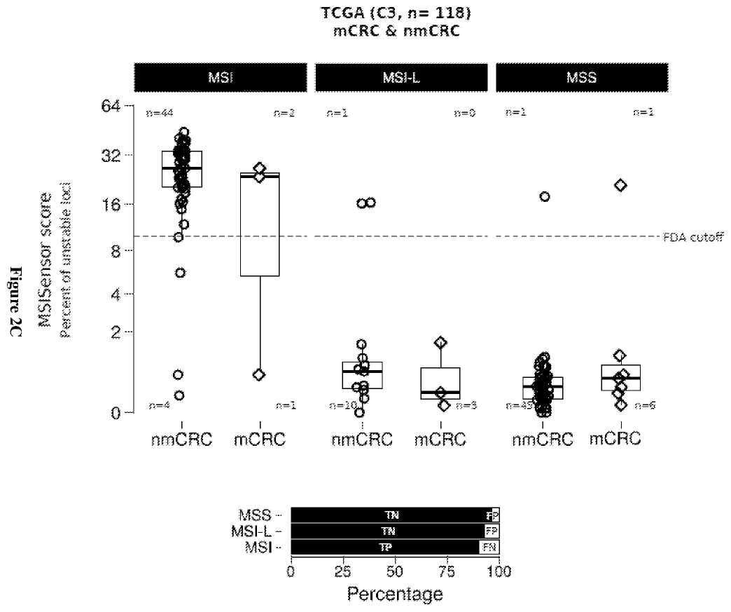

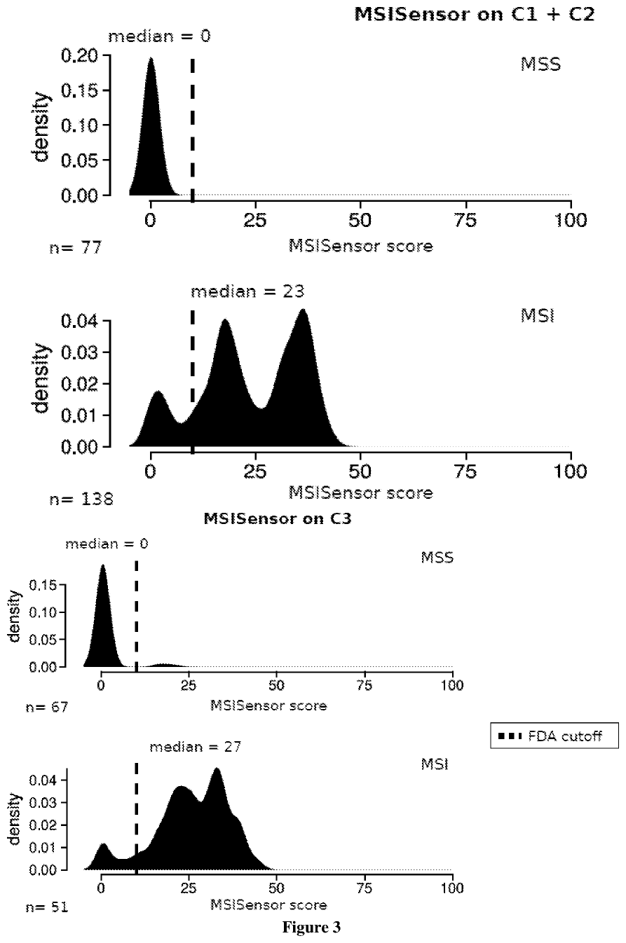

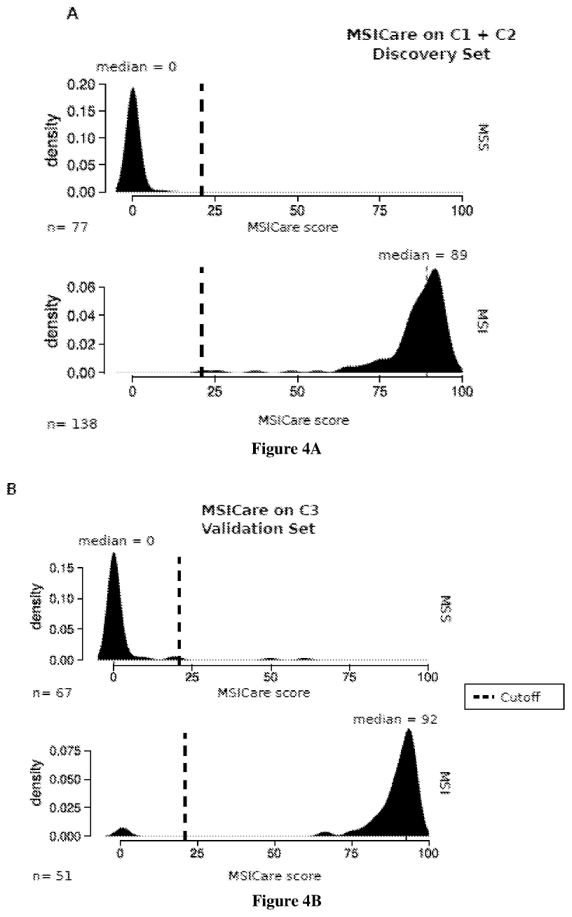

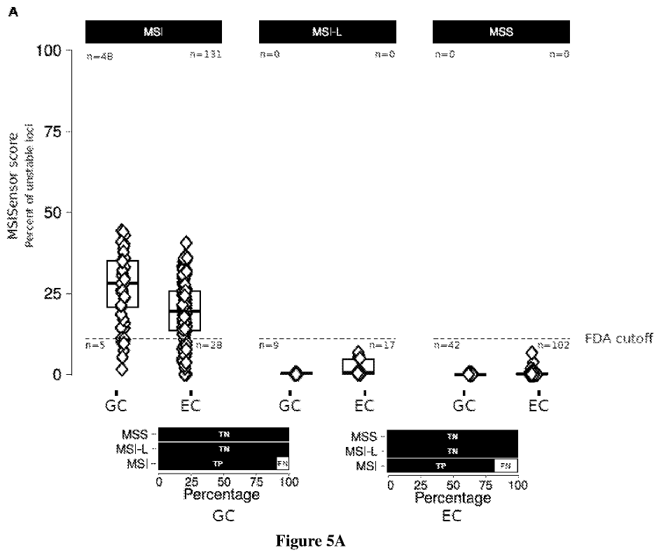

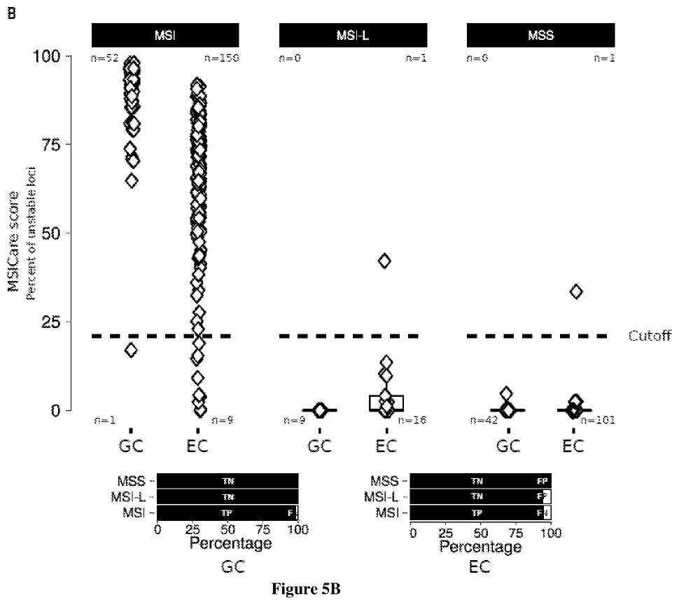

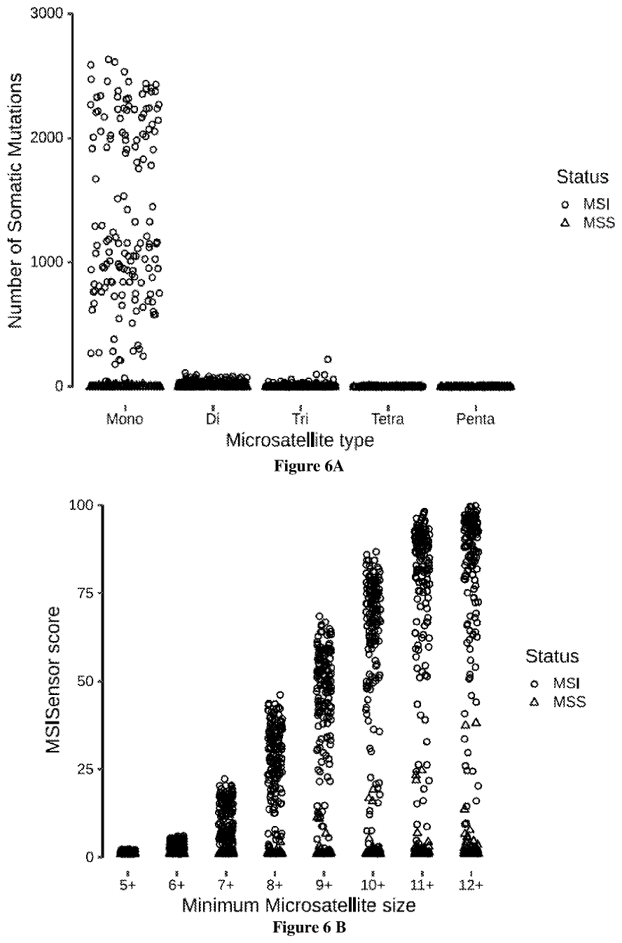

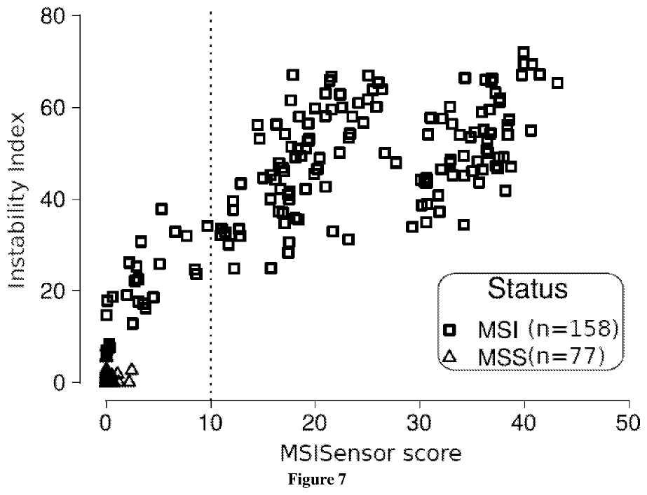

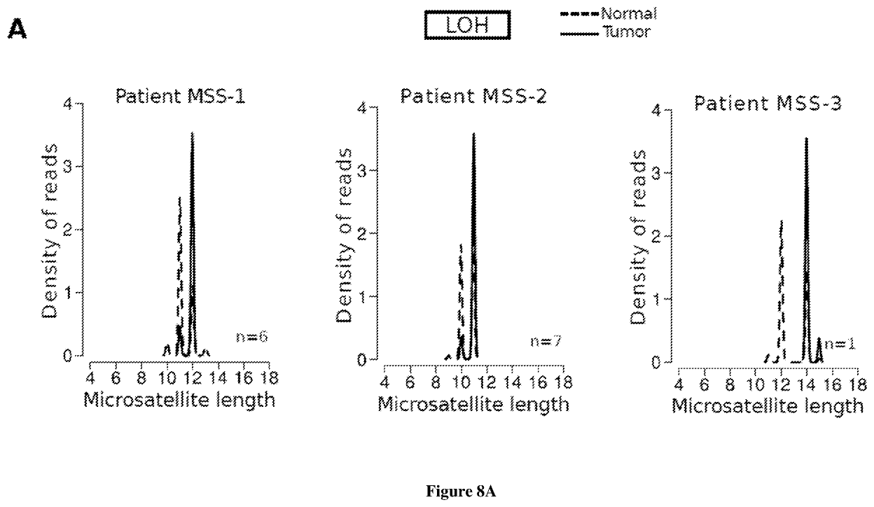

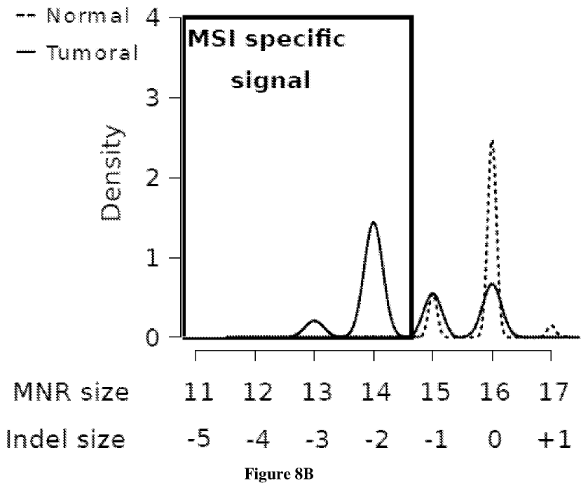

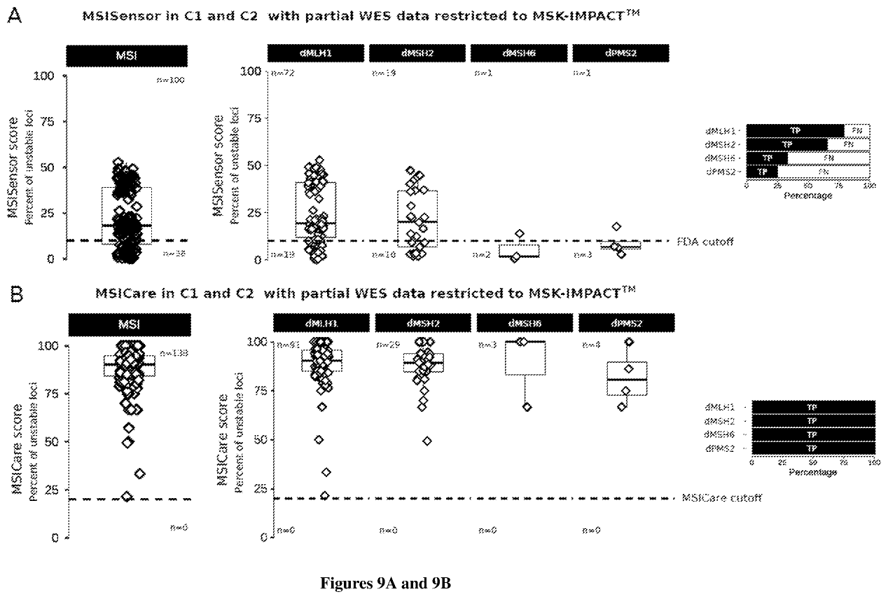

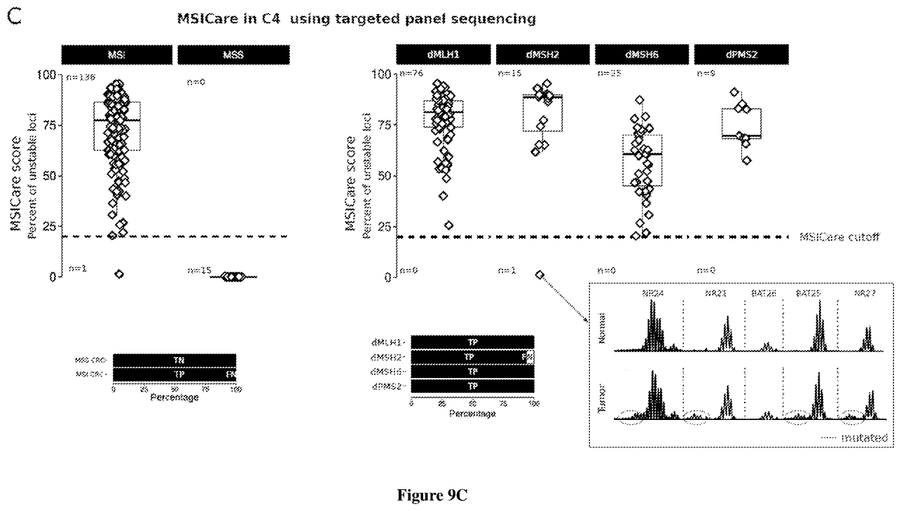

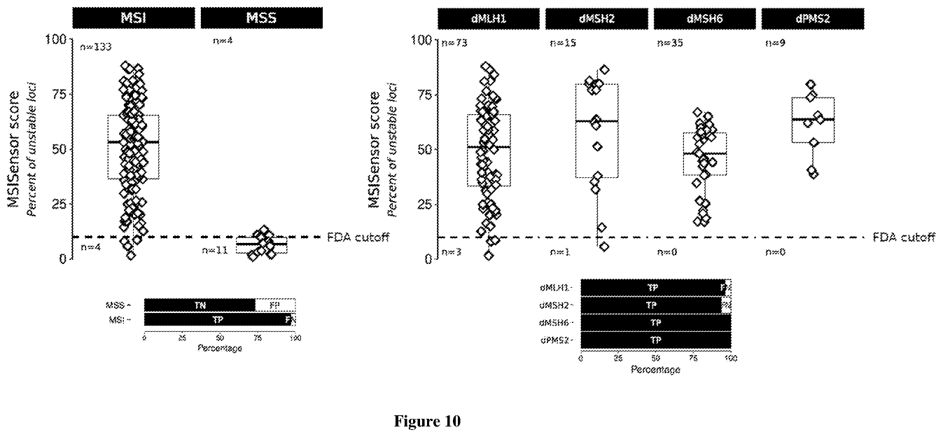

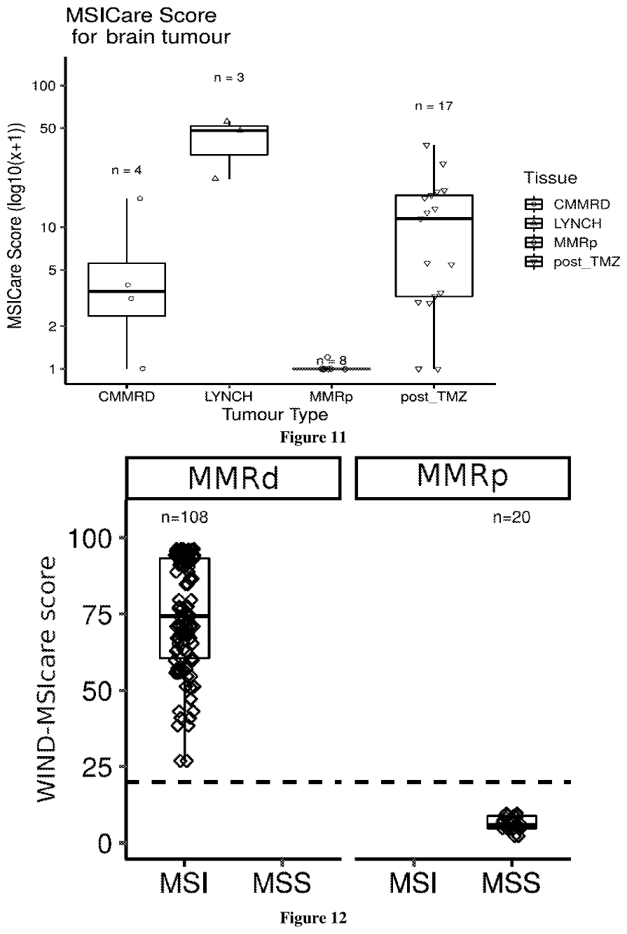

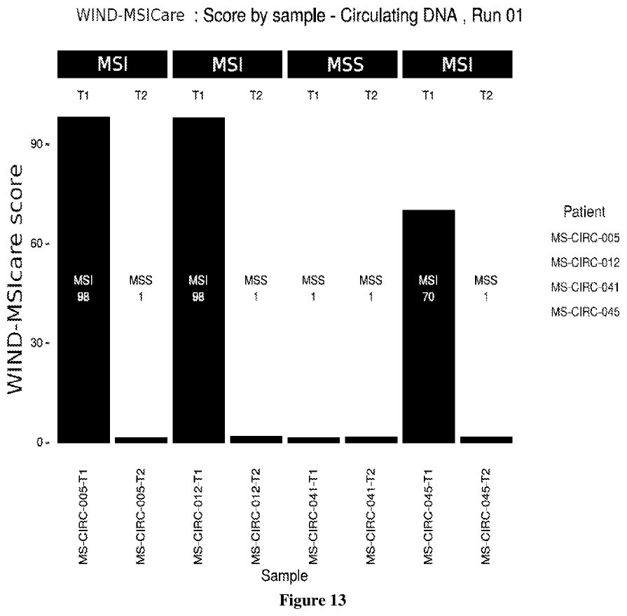

The clinical rationale and design of this study are presented in FIG. 1. One hundred and two patients with mCRC (Cohort C1, FIG. 1) originated from two multicenter French clinical trials (NCT02840604 and NCT033501260) which accrued patients between May 2015 and November 2019.

NCT02840604 aimed to show that exome analysis is feasible in the routine care of patients, thereby improving access to targeted therapies and improving the detection of genetic cancer predisposition. Genomic sequencing (WES) was performed at the Georges-Francois Leclerc Cancer Center, Genomic and Immunotherapy Medical Institute, Dijon, France. Patients were eligible if they presented with a locally advanced, non-operable or metastatic cancer that had progressed during at least one line of systemic therapy. The NIPICOL trial (NCT033501260) involves treatment of MSI/dMMR mCRC patients with nivolumab (anti-PD-1) and ipilimumab (anti-CTLA-4). mCRC response to ICI was determined according to Response Evaluation Criteria in Solid Tumors (RECIST) (20). Twenty-six cases from the NCT033501260 cohort were treated with ICI. Of these, 23 were confirmed to be MSI/dMMR and 3 were identified later as MSS/pMMR following reassessment of their MSI and MMR status centrally. Genomic sequencing (WES) was performed by IntegraGen SA (Evry, France). All patients provided signed informed consent for the trials and genomic analysis. After giving consent, patients underwent consultation with a geneticist to explain the consequences of constitutional genetic testing. Following this consultation, the patient could accept or refuse to provide a blood sample for constitutional exome analysis. This trial protocol was approved by an institutional review committee and performed in accordance with the Declaration of Helsinki.

A historical retrospective cohort was also analyzed (cohort C2, FIG. 1). This comprised 25 patients with mCRC that were diagnosed between 1998 and 2016 in 6 French hospitals as MSI or dMMR (17), as well as 88 patients from the Saint Antoine Hospital, Paris, who were diagnosed between 1998 and 2007 as having MSI/dMMR nmCRC (21). Primary and/or metastatic tumor tissues for mCRC were analyzed by IntegraGen SA (Evry, France) using WES. All patients provided written consent and the study was approved by the institutional review boards/ethics committees of the participating centers.

We further evaluated and compared the performance of MSICare and MSISensor in a third independent tumor cohort (C3). This included 118 CRC patients whose MSI status was previously assessed by PCR using the Bethesda microsatellite panel and whose WES data was publicly available from the TCGA. All CRC patients with MSI-H (n=51) or MSI-L (n=14) and a similar proportion of patients with MSS (n=53) were included (22). C3 also included 382 extra-colonic tumors from the TCGA with a relatively high incidence of MSI, i.e. gastric (53 MSI-H, 9 MSI-L, 42 MSS) and endometrial (159 MSI-H, 17 MSI-L, 102 MSS) cancers.

A new retrospective cohort was examined (cohort C4) using targeted NGS because WES is not routinely used in clinical care (see below for details). C4 was retrospective, non-consecutive, assembling 152 new patients from the Saint Antoine Hospital, Paris, and the Lille University Hospital, who were previously diagnosed as having MSI/dMMR or MSS/pMMR CRC (137 MSI, 15 MSS) using MSI PCR and IHC. dMMR/MSI CRC cases from the Saint-Antoine Hospital were previously diagnosed as being dMMR/MSI between 1998 and 2021 regardless of the MMR defect detected in the tumor. Both tumor and non-tumor DNA material was available for these cases and they were not previously analyzed by WES (no overlap with the C2 cohort). dMMR/MSI CRC cases from the Lille University Hospital with material available (biopsy or surgical resection) from 2016 to 2021 displayed isolated loss of MSH6 or PMS2 expression. They were selected to further evaluate the performance of MSICare for identifying MSI in these rare dMMR/MSI CRC settings, in particular for MSH6-deficient CRC which are known to be more difficult to diagnose²¹.

To investigate MSI phenotype in pan-cancer, and additional public retrospective cohort of non-CRC patients (cohort C5) was also analysed using WES. This cohort comprises 34 patients whose WES data was publicly available from the TCGA. This cohort includes Breast invasive carcinoma (BRCA, n=8), Cervical squamous cell carcinoma and endocervical adenocarcinoma (CESC, n=7), Esophageal carcinoma (ESCA, n=3), Head and Neck squamous cell carcinoma (HNSC, n=3), Acute Myeloid Leukemia (LAML, n=4), Lung squamous cell carcinoma (LUSC, n=4) and Skin Cutaneous Melanoma (SKCM, n=5). These patients were selected if they have mutational signatures implying a DNA repair deficiency, more specifically either MMR, MMR/POLE or POLE signature. MSICare and MSIsensor was then used comparatively on the sequencing data taking into account both normal and tumor tissues.

Moreover, to investigate the performance of MSIcare in non-CRC samples, a cohort (C6) of 32 patients with brain tumors were also studied. This cohort includes patient from the Institut du Cerveau-ICM, Paris, and patient were stratified into 4 groups: patients with Constitutional Mismatch Repair Deficiency (CMMRD, n=4), Lynch syndrome (Lynch, n=3), Glioblastoma recurrence after Temozolomide treatment (post_TMZ, n=17) and MMR proficient Glioblastoma (n=8). MSICare were applied in this tumor location which is known to be difficult to diagnose.

Finally, in order to assess the feasibility of MSI diagnosis using liquid biopsy, blood samples of a pilot of 4 patients with metastatic CRC were analysed (Cohort C7). These patients were initially included in the NIPICOL trial (C1, NCT033501260) and among the 4 patients, 3 were dMMR/MSI and 1 pMMR/MSS using the reference methode IHC and MSI-PCR respectively. Tumor circulating DNA (ctDNA) sequencing data was analysed using MSIcare normal-sample-free (WIND-MSIcare, see below).

All patients provided written consent and the study was approved by the institutional review boards/ethics committees of the participating centers.

### Samples

In the prospective cohort (C1, clinical trials NCT02840604 and NCT033501260), the mCRC samples were formalin fixed and paraffin-embedded (FFPE) and were comprised of either the primary or metastatic tumor tissue. In the retrospective cohort (C2), all nmCRC samples (n=88) were stored at −80° C. until DNA extraction. For mCRC patients (n=25), both the primary tumor and metastasis preserved in FFPE (n=45; 25 primary tumors and 20 metastasis) were collected and analyzed whenever possible in order to provide a more complete description of this rare CRC subtype. For the public retrospective cohort (C3), frozen tissue samples were collected from the primary tumor sites (colorectum, stomach, endometrium) as described (22). In the C4 retrospective cohort, CRC samples (N=152; primary or metastases) and matched non tumor samples were either FFPE (N=87) or frozen (N=65) in order to appreciate the feasibility of the MSICare method under various technical conditions and qualities of DNAs. Matched normal colonic mucosa samples were considered in all cohorts to perform NGS-based MSI.

All CRC samples from C4 in this study were centrally reassessed in expert centers involved in this study (Saint-Antoine hospital, Paris, France and Lille University Hospital, France) for dMMR status using immunohistochemistry (IHC) and for MSI using polymerase chain reaction (PCR) as previously described¹⁴⁻¹⁶.

For the public retrospective non-CRC cohort (C5), frozen tissue samples were collected from the primary tumor sites as described²⁴. And for the brain tumor cohort (C6) all samples were FFPE, and MMR status was assessed using IHC. For this cohort, normal mucosa or blood DNA was used as a matched normal sample.

Finally, for liquid biopsy samples (C7), blood DNA was extracted from plasma of patients with metastatic CRC treated with Immune checkpoint Inhibitors. Two time points T1 and T2 were performed, before treatment and 3 months after treatment respectively.

### Immunohistochemistry and MSI-PCR

All CRC samples from C1 and C2 used in this post-hoc study were centrally reassessed in our expert center (Saint-Antoine hospital, Paris France) for dMMR status using immunohistochemistry (IHC) and for MSI using polymerase chain reaction (PCR) as previously described (13-15).

### MSISensor End Points

False negatives from C1 and C2 were defined as samples initially diagnosed as MSI or dMMR using MSI-PCR and IHC, respectively, but showing a negative MSISensor Score (≤ 10%) when considering the complete exome data (18, 19). This was done by central assessment at the Georges-Francois Leclerc Cancer Center, Dijon, and at the Saint-Antoine Hospital, Paris. The sensitivity of MSISensor was calculated as the percentage of true-positive cases amongst the total of true-positive and false-negative cases.

ImpactTM gene panel (C1, C2, C3)19, 20; (ii) or MSIDIAG microsatellite panel of markers (See below) following targeted sequencing of tumors (C4). This was done by central assessment at the Saint-Antoine Hospital, Paris (C1, C2, C3, C4), at the Georges-Francois Leclerc Cancer Center, Dijon (C1), or at the Lille University Hospital (C4). The sensitivity of MSISensor was calculated as the percentage of true-positive cases amongst the total of true-positive and false-negative cases.

Whole Exome Sequencing and NGS-Based MSI Diagnosis with MSISensor

For the prospective (C1) and retrospective (C2) cohorts, the WES procedure was performed as recommended by the manufacturer (SureSelect Human Exon Kit v5, 75 MB; Agilent, Les Ulis, France) and as previously described (23). For metastatic tumor samples, the generated reads were mapped to the reference genome hg38 (GRCh38), while for the retrospective non-metastatic samples the reads were mapped onto hg19 (GRC37). MSISensor was used at the default setting to evaluate the mutation status of microsatellites from the WES data (19).

### Implementation of the Optimized NGS-Based MSICare Method to Increase the Sensitivity of MSI Detection in CRC and in Other Tumors

A new method (MSICare) was developed to optimize the detection of MSI based on comparison of the read distribution between normal and tumor samples from WES data. Mononucleotide repeats (MNR) with a length ≥12 base pairs (bp) were considered for analysis only if they were covered by at least 20 mapping reads in both normal and tumor samples. The total number of reads covering each candidate MNR was then normalized (arbitrary value of 100) in tumor and matched healthy tissue. For each MNR, the normalized number of reads in healthy tissue was subtracted from the normalized number of reads in tumor tissue [ΔRatio=% Tumor−% Normal] to generate an MSI index (MSI signal, MSIg) corresponding to the sum of ΔRatio values for all candidate MNR. The ΔRatio value was then adjusted by estimating the tumor purity (TP) for each tumor sample, with the estimated TP corresponding to the median value of the MSI signal for all MNR with a length ≥14 bp covered by at least 30 reads in tumor and 20 reads in normal tissue. The adjusted value for ΔRatio was then used to classify a given MNR as wild type (ΔRatio-adjusted=ΔRatio×Estimated TP<50%) or mutated (ΔRatio-adjusted=ΔRatio×Estimated TP≥50%) given that observed microsatellite mutations can be either heterozygous or homozygous in primary tumor samples. Finally, the MSICare score for tumor samples corresponds to the percentage of microsatellites that were mutated amongst the total number of microsatellites analyzed using this approach. The scripts and documentation are available through Github at https://github.com/MSI.CRSA/MSICare.

### MSICare Cutoff Determination

A cutoff value for MSICare was estimated in order to optimize the differentiation of MSI from MSS samples in the different cohorts. This was done using the cutpointr package (version 1.0.32), which estimates optimal cutoff points in binary classification tasks and validates their performance using bootstrapping. A cutoff point of 20 was determined using a discovery set of 77 MSS and 138 MSI (C1+C2; CRC, Discovery set) and then applied to a validation set of MSI (C3; CRC and non-CRC, Validation set) from public TCGA data (see the Results section for further details). The same cutoff was tested again to test MSICare for identifying MSI in the same cohorts of CRC patients when considering only partial WES data restricted to the MSK-ImpactTM gene panel.

Diagnosing MSI in CRC with MSICare Following Targeted Sequencing of Paired Tumoral and Normal Mucosa Samples with an Optimized Panel of Microsatellite Markers

From the viewpoint of clinical application, MSI test is important not only in Whole exome sequencing, but also in panel testing. The performance of MSICare as compared to MSISensor was assessed again in the additional independent, multicenter CRC cohort (C4) using the same cutoff. Sequencing of this cohort on paired tumor and normal mucosa samples was performed using an optimized targeted panel of microsatellite markers, namely MSIDIAG. This panel includes 441 mononucleotide repeats which have been selected among the MNR harboring a size of 12 bp or more whose instability was exclusively observed in MSI tumor samples from C1, C2 and C3 following WES (low frequency of somatic mutations in MSS CRC; chi-squared test with p-value <0.05). After capture and sequencing, reads were mapped to the Human genome build (hg38) with depth of coverage comprised between 100× and 500×. The diagnosis of MSI was assessed using MSISensor or MSICare procedure exactly as this was performed previously from WES data in C1, C2 and C3 (see above).

### Implementation of WIND-MSICare for Detecting MSI Status in CRC Using Normal-Sample-Free (Tumor Only) DNA Sequencing Data on Both Solid and Liquid Biopsy.

To detect MSI without matching normal samples, a normal polymorphic zone was identified for each repeat using a database of 764 normal samples. Mononucleotide repeats (MNR) with a length ≥12 base pairs (bp) were considered for analysis only if they were covered by at least 20 mapping reads in tumor samples. The total number of reads covering each candidate MNR in the tumor was then normalized (arbitrary value of 100). Then, from the tumoral sample, only mutated repeats observed outside this normal polymorphic zone was considered. Outside this polymorphic zone, the number of reads in the normal sample were considered equal to zero [ΔRatio=% Tumor outside polymorphic zone] for each MNR. In this context, the several steps of the MSICare method (see above), has been done taking this into account.

An MSI index (MSI signal, MSIg) were genetated and corresponded to the sum of ΔRatio values for all candidate MNR. The ΔRatio value was then adjusted by estimating the tumor purity (TP) for each tumor sample, with the estimated TP corresponding to the median value of the MSI signal for all MNR with a length ≥14 bp covered by at least 30 reads in tumor. The adjusted value for ΔRatio was then used to classify a given MNR as wild type (ΔRatio-adjusted=ΔRatio×Estimated TP<50%) or mutated (ΔRatio-adjusted=ΔRatio×Estimated TP≥50%) given that observed microsatellite mutations can be either heterozygous or homozygous in primary tumor samples. Finally, the WIND-MSICare (Without Including Normal DNA) score for tumor samples corresponds to the percentage of microsatellites that were mutated amongst the total number of microsatellites analyzed using this approach.

This method was applied to patient tumor from C4 cohort (solid samples) and also on liquid biopsy (ctDNA) of a pilot of 4 patients (C7) displaying metastatic CRC. This last cohort was sequenced using the MSIDIAG panel and reads were mapped to the Human genome build (hg38) with a depth of coverage comprised between 3000× and 5000× in order to make the annalysis the most sensitive.

### Results

Frequent Misdiagnosis of MSI with MSISensor in Both mCRC and nmCRC

All CRC samples from C1 and C2 were centrally reassessed for MSI and dMMR status using the gold standard reference methods of pentaplex PCR and IHC (FIG. 1). MSISensor confirmed the status of 77 MSS/pMMR mCRC from the prospective C1 cohort (FIG. 1 and FIG. 2A; MSISensor score≤10%). However, it failed to confirm the status of 4 of the 25 MSI/dMMR mCRC samples (FIG. 2A; MSISensor score≤10%). The frequency of misdiagnosis in C1 was therefore 16% (N=4/25; sensitivity 84%, 95% CI: 69%-99%).

The sensitivity of MSISensor was further assessed in 25 mCRC patients with MSI/dMMR from the retrospective C2 cohort (FIG. 1). In mCRC, the frequency of misdiagnosis was even higher at 32% (N=8/25, 32%; sensitivity 68%, 95% CI: 49.3%-86.7%) (FIG. 2B). In 88 nmCRC patients with MSI/dMMR from the C2 cohort, misdiagnosis occurred in 9% (N=8/88, 9%; sensitivity 91%, 95% CI: 85%-97%) (FIG. 2B). A similar performance for MSISensor was observed with MSK-IMPACTTM (see Materials and Methods for details). The overall number of false negative cases detected amongst MSI/dMMR CRC was very similar for all 3 versions of the MSK panel used to determine the MSISensor score (data not shown). The sensitivity of MSISensor was also assessed in the public C3 cohort of CRC patients that included both nmCRC and mCRC (FIG. 1). The frequency of missed diagnoses was again very similar at 9.8% (N=5/51), giving a sensitivity of 90% (95% CI: 82%-98%) in patients with MSI/dMMR CRC. This included one misdiagnosed case of mCRC (1/3, 33%) (FIG. 2C). MSISensor confirmed the status of all but 2 MSS/pMMR mCRC from C3, thus indicating the major limitation of this method was its lack of sensitivity. The overall performance of MSISensor in the C1 cohort compared to the C2 and C3 cohorts is shown in Table 1A.

### Identifying Weaknesses and Limits of MSISensor by Deciphering the MSI Genomic Signal of DNA Repeats in CRC

A density plot was created to show fluctuations in the MSISensor score for the 3 patient cohorts analyzed in this study (FIG. 3). The MSI/dMMR and MSS/pMMR status of all samples in cohorts C1 and C2 were pooled as these had previously been validated centrally. CRC samples from the public cohort C3 were considered separately since we were unable to independently confirm the status of these tumors using IHC and MSI-PCR. The density profiles clearly highlight the lack of sensitivity of MSISensor for the detection of MSI in dMMR CRC, as already shown above for the 3 cohorts (FIG. 2 and Table 1).

We next hypothesized that it should be possible to improve the detection of MSI in CRC by modifying certain parameters in the analysis of NGS data. In support of this, WES analyses revealed that MSISensor lacked sensitivity because: (i) MNR sequences were by far the most unstable category of microsatellites in dMMR colon tumors and therefore better at distinguishing MSI from MSS CRC than other types of repeat used by MSISensor (e.g. di-, tri-, tera-, penta-) (FIG. 6A); (ii) the ability of MNR to discriminate between MSS and MSI colon tumors was dependent upon their length, with long MNR of ≥12 bp found to be the most discriminating compared to other microsatellites used by MSISensor (FIG. 6B); (iii) MSISensor was not suitable for detecting MSI in CRC samples with an estimated TP of less than 30-40%. This is an important limitation to the sensitivity of MSISensor in primary MSI CRC due to the often high levels of contamination with non-tumor and inflammatory pMMR/MSS cells (FIG. 7) (see also our review (24) and original publications for further details 14, 15, 21, 23, 25). WES analyses also revealed that MSISensor lacks specificity for two reasons. Firstly, the MSISensor computational tool confused the true MSI signal with allelic losses (LOH) for some of the MNR. LOH occurs frequently in MSS colon tumors with high levels of chromosomal instability (FIG. 8A). Secondly, stuttering by DNA polymerase during the PCR reaction occurs frequently at microsatellites and in particular at long MNR. A misdiagnosis of MSI can therefore occur when small 1 bp deletions in these microsatellites are considered by MSISensor to represent MSI (FIG. 8B).

### Designing and Validating MSICare to Improve NGS-Based Detection of MSI in CRC

To avoid the abovementioned pitfalls of MSISensor, we next designed a new computational tool referred to as MSICare to accurately detect MSI in CRC based on analysis of their WES profile. In contrast to MSISensor, MSICare identifies true MSI signals defined as somatic deletions of at least 2 bp in length that occur in long MNR (≥12 bp) in DNA from dMMR cancers but not in DNA from paired normal tissue (see Materials and Methods for further details). A Receiver Operating Characteristic (ROC) curve was constructed assuming binary classification of the MSICare score. This revealed a perfect discrimination between dMMR and pMMR CRC in C1 and C2, with 100% sensitivity and 100% specificity when using a cut-off value of 20% (FIG. 4A and data not shown). dMMR/MSI samples showed a mean MSICare score above 80% with little dispersion around this value, whereas the mean MSICare score of pMMR/MSS samples was below 10%. MSICare thus appears to be very effective in discriminating MSI from MSS CRC cases. The high level of discrimination achieved using the cut-off value of 20% was validated in the public C3 cohort (FIG. 4B right panel) and led us to correct 3 cases amongst 5 that were true MSI that showed false negative status with MSISensor. Of interest, the two remaining CRC samples with negative MSISensor status that were previously classified as MSI by PCR according to TCGA remained unambiguously MSS with MSICare. Detailed analysis of the exomic profile of both these tumors revealed very few mutations in microsatellites located within coding regions of known MSI target genes (data not shown). Furthermore, one of the samples showed pMMR according to TCGA, thus suggesting an equivocal MSI status. The overall performance of MSICare in the C1 cohort compared to the C2 and C3 cohorts is shown in Table 1B.

We analyze the performance of MSICare taking into account the nature of the dMMR defect in tumors. The results indicate that the sensitivity of this test remain optimal in MSH6-deficient or PMS2-deficient colon tumors from this cohort (sensitivity 100%).

MSICare is Likely to have Better Performance than MSISensor for the Detection of MSI in Gastric and Endometrial Tumors

The performance of MSISensor for the detection of MSI was assessed in two other primary cancer types that frequently show an MSI phenotype, namely gastric cancer (GC) and endometrial cancer (EC). Investigation of the available WES data for GC and EC from the TCGA revealed a much better performance for MSICare in the detection of MSI as compared to MSISensor (FIG. 5A and B and Table 1B).

### Confirmation of the Performance of MSICare in Detecting MSI in CRC Using Targeted NGS

Because WES is not routinely used in clinical care, we finally aimed to confirm the high performance of MSICare for detecting MSI following targeted sequencing of CRC samples as compared to paired normal mucosa samples. This was first done in C1 and C2, considering only the microsatellites included in the restricted MSK-IMPACTTM gene panel (see Materials and Methods for details). The overall number of false negative cases detected amongst MSI/dMMR from these cohorts under these conditions was important with MSISensor for all 3 versions of MSK-IMPACTTM (FIG. 9A), particularly in MSH6 and PMS2 deficient settings (sensitivity 28.6%, 95% CI: 4.9%-62%) (FIG. 9A). By, contrast, the performance of MSICare remained optimal under the same conditions (sensitivity 100%) (FIG. 9B).

Next, we generated an optimally designed panel of 441 mononucleotide repeats (length≥12 pb and unstable in MSI tumors; See Methods for Details) called MSIDIAG. Using this panel we confirmed that, in the C4 cohort including 152 patients (137 MSI, 15 MSS) and that was enriched in CRC with MSH6 (35 patients) or PMS2 (9 patients) deficiency, MSICare still optimally detected MSI in CRC regardless of MMR defect (Sensitivity 99.3%, 95% CI: 97.8%-100.7%; Specificity 100%) (FIG. 9C) whereas MSISensor remained less sensitive while becoming unspecific expectedly (Sensitivity 97.1%, 95% CI: 92.7%-99.2%; Specificity 73.3%, 95% CI: 44.9%-92.2%) (FIG. 10).

### Comparative Analysis of MSICare and MSIsensor on Patients Displaying DNA Repair Defect Signature in Pan-Cancer

Among the 34 TCGA pan-cancer patients displaying mutational signature associated to MMR, MMR/POLE or POLE, 22/34 were identified as MSI with MSICare (data not shown) whereas 14/34 were identified as MSI with MSIsensor (data not shown). In Breast invasive carcinoma (BRCA) and Esophageal carcinoma (CESC) all patients with MMR mutational signature are classed MSI by MSICare, and patients with POLE signature are identified as MSS (data not shown). Using MSIsensor, in these 2 cancers types, 5/7 of the patitent identified as MSS (MSIsensor score <10) display MMR mutational signature and 2/7 display POLE signature (data not shown). This suggest that MSICare results seems to correlate well with MMR mutational signature in breast invasive and Esophageal carcinoma.

### Assessment of MSI Level in Brain Tumors Using MSICare

For brain tumor patients, we observed that microsatellite instability level was higher in CMMRD (n=4), LYNCH (n=3) and post_TMZ (n=17) MMR-deficient brain tumor samples than in MMR proficient brain tumor samples (n=8) using MSICare whereas this was not the case with MSISensor (FIG. 11). These results suggest that MSIcare can be used to diagnose MSI in brain tumors. However, additional experiments are required to define the optimal cutoff value since, expectedly, MSI/dMMR brain tumor samples displayed a mild MSI phenotype as compared to MSI CRC for instance (we remind that MSI PCR is unable to detect MSI in brain tumors).

### MSIcare Diagnosis in CRC Normal-Sample-Free Solid Tumor

The MSICare method without referencing to matching normal DNA was applied to detect MSI in a series of 128 colorectal samples from the C4 cohort of which 108 were MMRd/MSI and 20 were MMRp/MSS using IHC and PCR MSI, respectively. All samples were classified correctly using this approach (FIG. 12), highlighting that this new version of MSICare, namely WIND-MSICare, is likely to be as sensitive as MSICare to detect MSI in CRC. Additional experiments are in progress to investigate the performances of WIND-MSICare in pan-cancer.

### MSIcare Diagnosis in Tumor Circulating DNA

WIND-MSICare was tested again to detect MSI in circulating tumor DNAs extracted from the blood of patients with metastatic CRC (3 MSI, 1 MSS). In this pilot study, this algorithm was able to detect MSI in the 3 samples from patients with MSI CRC before they received ICI therapy (FIG. 13). In contrast, it failed to detect MSI in the patient with MSS CRC, expectedly, and also in the 3 samples from patients with MSI CRC after they received ICI therapy (FIG. 13). Even if they've been obtained only on a small series of patients, these results indicate that WIND-MSICare is likely to be available for detecting MSI in the plasma of MSI CRC patients. Additional results are required to investigate its performance in patients with non metastatic MSI CRC and/or patients with metastatic or non metastatic non-colorectal cancer.

### CONCLUSION

Several publications have recently highlighted the potential of NGS for the detection of MSI in human cancers by using distinct computational algorithms (18, 19, 26-29). Amongst these, MSISensor has received FDA approval and is used to guide the prescription of ICI therapy in patients with metastatic cancer, regardless of the primary location of the tumor. MSISensor has been tested on advanced solid cancers including a large number of CRC. However, the performance of this NGS-based test has yet to be evaluated in a large series of CRC previously assessed for MSI/dMMR status using the reference PCR and IHC methods. The accuracy of MSISensor is especially important for patients deemed as MSI/dMMR mCRC and subsequently treated with ICI. In this study the inventors provide clear evidence that MSISensor lacks sensitivity for the detection of MSI. This was shown in large cohorts of mCRC and nmCRC samples that were previously confirmed as MSI/dMMR or MSS/pMMR by IHC and MSI-PCR methods performed in large, specialized test centers. These results are of particular clinical relevance for ICI therapy. They highlight that in a prospective cohort of MSI mCRC patients, the consideration of results from MSISensor alone in the absence of MSI-PCR and IHC testing would have led to approximately 16% of patients (4/25) not being offered ICI treatment. Of the 4 patients not detected by MSISensor, 3 were found to be responsive to treatment. Lack of sensitivity for the detection of MSI in nmCRC, as shown here in a large retrospective patient cohort, can also have other adverse clinical consequences such as the failure to detect Lynch syndrome. From these findings they conclude there is an urgent need to change the NGS-based criteria for the identification of MSI in CRC. The present results in CRC patients are consistent with those of another study that found NGS was unable to detect MSI in dMMR tumors from two prostate cancer patients who displayed prolonged positive response to ICK blockade therapy (30). Both tumors showed a high mutational burden and a high density of intratumoral infiltration with CD3 cells. they therefore extrapolate that the low sensitivity of MSISensor for the detection of MSI is likely to apply to all tumors types, as suggested also by the analysis in the present study of gastric and endometrial tumors from the TCGA.

The new MSICare bioinformatic tool proposed here for the detection of MSI shows much better performance compared to MSISensor. It has 100% sensitivity and specificity compared to PCR-MSI in the CRC cohorts tested here, thus matching the performance of the gold standard IHC and MSI-PCR methods. Importantly, it detected MSI in 4 mCRCs that were not initially detected by MSISensor, 3 of which showed a positive response to immunotherapy. As an expert center for the analysis of MSI in clinical oncology, they have optimized this bioanalytic tool so that MSI detection in tumor DNA is highly sensitive while remaining specific. The use of MSICare makes it possible to diagnose MSI in CRC that is highly contaminated with stromal tissue, which is frequently the case in MSI primary tumors. Of note, this new algorithm shows the same performance for both FFPE and frozen primary or metastatic tissue samples regardless of their primary MMR defect in MLH1, MSH2, MSH6 or PMS2, suggesting that either tissue material can is suitable for the analysis.

It was notably checked to be relevant, in contrast to MSISensor, for identifying MSI in MSH6-deficient CRC in which MSI is more difficult to diagnose and in PMS2-deficient CRC which are scarce. Importantly, and in contrast to MSISensor again, its performance for assessment of MSI remained optimal when tested with the full or partial exome data restricted to the MSK-ImpactTM panel of markers. MSICare also showed great performances when exploited with an optimally designed set of microsatellite markers following targeted sequencing of tumor samples (MSIDIAG). The outstanding diagnostic performance of MSICare to detect MSI with different sequencing strategies on independent series of CRC validates with a high level of evidence the relevance of this method to detect MSI in CRC. The MSIDIAG panel includes mononucleotide repeats that are of particular interest for detecting MSI in tumor DNA and it is therefore recommended to use this panel with MSICare in targeted sequencing analyses for optimal sensitivity of this assay. In summary, these data establish that MSICare has the potential to become a new NGS-based international reference method for the determination of MSI phenotype in CRC from WES or targeted NGS using home-made or FDA-approved panels. It should become very useful for translational research, clinical trials and in routine clinical practice in the management of CRC patients, especially as MSI is becoming an indispensable theranostic biomarker in the metastatic setting.

In summary, MSICare would be very useful for routine clinical practice in the management of CRC patients and others cancers, especially as MSI is becoming an indispensable theranostic biomarker in the metastatic setting.

### REFERENCES

Throughout this application, various references describe the state of the art to which this invention pertains. The disclosures of these references are hereby incorporated by reference into the present disclosure.
1 Aaltonen L A, Peltomaki P, Leach F S, et al. Clues to the pathogenesis of familial colorectal cancer. Science 1993; 260:812-6.
2. Fishel R, Lescoe M K, Rao M R, et al. The human mutator gene homolog MSH2 and its association with hereditary nonpolyposis colon cancer. Cell 1993; 75:1027-38.
3. Thibodeau S N, Bren G, Schaid D. Microsatellite instability in cancer of the proximal colon. Science 1993; 260:816-9.
4 Ionov Y, Peinado M A, Malkhosyan S, et al. Ubiquitous somatic mutations in simple repeated sequences reveal a new mechanism for colonic carcinogenesis. Nature 1993; 363:558-61.
5 Lothe R A, Peltomaki P, Meling G I, et al. Genomic instability in colorectal cancer: relationship to clinicopathological variables and family history. Cancer Res 1993; 53:5849-52.
6 Llosa N J, Cruise M, Tam A, et al. The vigorous immune microenvironment of microsatellite instable colon cancer is balanced by multiple counter-inhibitory checkpoints. Cancer Discov 2015; 5:43-51.
7 Marchisa L, Svrcek M, Collura A, et al. The Balance Between Cytotoxic T-cell Lymphocytes and Immune Checkpoint Expression in the Prognosis of Colon Tumors. J Natl Cancer Inst 2018; 110.
8 Le D T, Uram J N, Wang H, et al. PD-1 Blockade in Tumors with Mismatch-Repair Deficiency. N Engl J Med 2015; 372:2509-20.
9 Overman M J, Lonardi S, Wong K Y M, et al. Durable Clinical Benefit With Nivolumab Plus Ipilimumab in DNA Mismatch Repair-Deficient/Microsatellite Instability-High Metastatic Colorectal Cancer. J Clin Oncol 2018; 36:773-779.
10. Overman M J, McDermott R, Leach J L, et al. Nivolumab in patients with metastatic DNA mismatch repair-deficient or microsatellite instability-high colorectal cancer (CheckMate 142): an open-label, multicentre, phase 2 study. Lancet Oncol 2017; 18:1182-1191.
11. Le D T, Kim T W, Van Cutsem E, et al. Phase I I Open-Label Study of Pembrolizumab in Treatment-Refractory, Microsatellite Instability-High/Mismatch Repair-Deficient Metastatic Colorectal Cancer: KEYNOTE-164. J Clin Oncol 2020; 38:11-19.
12. Luchini C, Bibeau F, Ligtenberg M J L, et al. ESMO recommendations on microsatellite instability testing for immunotherapy in cancer, and its relationship with PD-1/P D-L1 expression and tumour mutational burden: a systematic review-based approach. Ann Oncol 2019; 30:1232-1243.
13. Suraweera N, Duval A, Reperant M, et al. Evaluation of tumor microsatellite instability using five quasimonomorphic mononucleotide repeats and pentaplex PCR. Gastroenterology 2002; 123:1804-11.
14 Buhard O, Cattaneo F, Wong Y F, et al. Multipopulation analysis of polymorphisms in five mononucleotide repeats used to determine the microsatellite instability status of human tumors. J Clin Oncol 2006; 24:241-51.
15. Buhard O, Lagrange A, Guilloux A, et al. HSP110 T17 simplifies and improves the microsatellite instability testing in patients with colorectal cancer. J Med Genet 2016; 53:377-84.
16 Svrcek M, Lascols O, Cohen R, et al. MSI/MMR-deficient tumor diagnosis: Which standard for screening and for diagnosis? Diagnostic modalities for the colon and other sites: Differences between tumors. Bull Cancer 2019; 106:119-128.
17. Cohen R, Hain E, Buhard O, et al. Association of Primary Resistance to Immune Checkpoint Inhibitors in Metastatic Colorectal Cancer With Misdiagnosis of Microsatellite Instability or Mismatch Repair Deficiency Status. JAMA Oncol 2019; 5:551-555.
18. Middha S, Zhang L, Nafa K, et al. Reliable Pan-Cancer Microsatellite Instability Assessment by Using Targeted Next-Generation Sequencing Data. JCO Precis Oncol 2017; 2017.
19. Niu B, Ye K, Zhang Q, et al. MSIsensor: microsatellite instability detection using paired tumor-normal sequence data. Bioinformatics 2014; 30:1015-6.
20. Eisenhauer E A, Therasse P, Bogaerts J, et al. New response evaluation criteria in solid tumours: revised RECIST guideline (version 1.1). Eur J Cancer 2009; 45:228-47.
21. Collura A, Lagrange A, Svrcek M, et al. Patients with colorectal tumors with microsatellite instability and large deletions in HSP110 T17 have improved response to 5-fluorouracil-based chemotherapy. Gastroenterology 2014; 146:401-11 el.
22. Hause R J, Pritchard C C, Shendure J, et al. Classification and characterization of microsatellite instability across 18 cancer types. Nat Med 2016; 22:1342-1350.
23. Jonchere V, Marisa L, Greene M, et al. Identification of Positively and Negatively Selected Driver Gene Mutations Associated With Colorectal Cancer With Microsatellite Instability. Cell Mol Gastroenterol Hepatol 2018; 6:277-300.
24. Duval A, Hamelin R. Mutations at coding repeat sequences in mismatch repair-deficient human cancers: toward a new concept of target genes for instability. Cancer Res 2002; 62:2447-54.
25. Dorard C, de Thonel A, Collura A, et al. Expression of a mutant HSP110 sensitizes colorectal cancer cells to chemotherapy and improves disease prognosis. Nat Med 2011; 17:1283-89.
26. Salipante S J, Scroggins S M, Hampel H L, et al. Microsatellite instability detection by next generation sequencing. Clin Chem 2014; 60:1192-9.
27. Kautto E A, Bonneville R, Miya J, et al. Performance evaluation for rapid detection of pan-cancer microsatellite instability with MANTIS. Oncotarget 2017; 8:7452-7463.
28. Vanderwalde A, Spetzler D, Xiao N, et al. Microsatellite instability status determined by next-generation sequencing and compared with P D-L1 and tumor mutational burden in 11,348 patients. Cancer Med 2018; 7:746-756.
29. Johansen A F B, Kassentoft C G, Knudsen M, et al. Validation of computational determination of microsatellite status using whole exome sequencing data from colorectal cancer patients. BMC Cancer 2019; 19:971.
30. Antonarakis E S, Piulats J M, Gross-Goupil M, et al. Pembrolizumab for Treatment-Refractory Metastatic Castration-Resistant Prostate Cancer: Multicohort, Open-Label Phase II KEYNOTE-199 Study. J Clin Oncol 2019:JCO1901638.

1-8. (canceled)

9. A method of diagnosing an MSI cancer in a patient in need thereof comprising i) extracting DNA from a tumoral sample and if available from a normal sample obtained from said patient, ii) sequencing a number N of mononucleotide repeats-MNR-sequences having a length of at least 12 nucleic acids in the DNA of the normal sample of said patient and the corresponding MNR in the DNA of the tumoral sample of said patient or sequencing a number N of mononucleotide repeats in the tumoral sample and having a length of at least 12 nucleic acids in the corresponding normal sample, iii) calculating the ΔRatio for each MNR depending if the normal sample is available or not, iv) calculating the tumor purity—TP—for the tumor sample, v) calculating the ΔRatio adjusted, vi) obtaining a MSICare score by doing the ratio of the number of MNR with a ΔRatio adjusted mutated on the total number of ΔRatio of the MNR and, vii) concluding that the patient in need thereof has a MSI cancer when the MSICare score obtained at the step vi) is superior than a calculated threshold value.

10. The method of diagnosing an MSI cancer in a patient in need thereof according to the claim 9, comprising i) extracting DNA from a tumoral and normal samples obtained from said patient, ii) sequencing a number N of mononucleotide repeats—MNR—sequences having a length of at least 12 nucleic acids in the DNA of the normal samples of said patient and the corresponding MNR in the DNA of the tumoral samples of said patients, iii) calculating the Ratio for each MNR, iv) calculating the tumor purity—TP—for the tumor sample, v) calculating the Ratio adjusted, vi) obtaining a MSICare score by doing the ratio of the number of MNR with a Ratio adjusted mutated on the total number of ΔRatio of the MNR and, vii) concluding that the patient in need thereof has a MSI cancer when the MSICare score obtained at the step vi) is superior than a calculated threshold value.

11. The method of diagnosing an MSI cancer in a patient in need thereof according to the claim 9, comprising i) extracting DNA from a tumoral sample obtained from said patient, ii) sequencing a number N of mononucleotide repeats—MNR—in the tumoral sample and having a length of at least 12 nucleic acids in the corresponding normal sample, iii) evaluating the normal polymorphic zones for the MNR, iv) evaluating the mutated MNR appearing in the tumor sample only outside the normal polymorphic zone of each MNR; v) calculating the ΔRatio for each MNR obtained from the tumoral sample; vi) calculating the tumor purity—TP—for the tumor sample, vii) calculating the ΔRatio adjusted, viii) obtaining a MSICare score by doing the ratio of the number of MNR with a ΔRatio adjusted mutated on the total number of ΔRatio of the MNR and, ix) concluding that the patient in need thereof has a MSI cancer when the MSICare score obtained at the step viii) is superior than a calculated threshold value.

12. The method according to claim 9, wherein the cancer is metastatic or not.

13. The method according to claim 9, wherein the cancer is a colorectal cancer, a gastric cancer or an endometrial cancer.

14. The method according to claim 9, wherein a further step of communicating the result to the patient is added.

15. A method of diagnosing a mutation in a MNR in a patient in need thereof comprising i) extracting DNA from a tumor and normal samples obtained from said patient, ii) sequencing a number N of mononucleotide repeats-MNR-sequences having a length of at least 12 nucleic acids in the DNA of the normal samples of said patient and the corresponding MNR in the DNA of the tumoral samples of said patients, iii) calculating the ΔRatio for each MNR, iv) calculating the tumor purity—TP—for the tumor sample, v) calculating the ΔRatio adjusted and vi) concluding that the MNR is wild type when the ΔRatio-adjusted is <50% concluding that the MNR is mutated when the ΔRatio adjusted is ≥50%.

16. A method for treating a cancer in a patient identified has having a MSI cancer according to a method for diagnosing a MSI cancer according to claim 9, comprising administering to said patient a therapeutically effective amount of radiotherapy, chemotherapy, immunotherapy or a combination thereof.
